SEQUENCE LISTING (56 sequences)

SEQ ID NO: 1 - DNA, 19 bp, synthetic construct
  source 1..19: mol_type = other DNA
caacgattcccagatcgaa

SEQ ID NO: 2 - DNA, 19 bp, synthetic construct
  source 1..19: mol_type = other DNA
cctcctaaagaggatataa

SEQ ID NO: 3 - DNA, 19 bp, synthetic construct
  source 1..19: mol_type = other DNA
ggaaattcctagaacataa

SEQ ID NO: 4 - DNA, 19 bp, synthetic construct
  source 1..19: mol_type = other DNA
ggattctgtattatggata

SEQ ID NO: 5 - DNA, 19 bp, synthetic construct
  source 1..19: mol_type = other DNA
cacaaagagaggattgcta

SEQ ID NO: 6 - DNA, 19 bp, synthetic construct
  source 1..19: mol_type = other DNA
ggcatgctgaagagacgaa

SEQ ID NO: 7 - DNA, 19 bp, synthetic construct
  source 1..19: mol_type = other DNA
cggcatgataacaaggtta

SEQ ID NO: 8 - DNA, 19 bp, synthetic construct
  source 1..19: mol_type = other DNA
gcttaaccctagttcacga

SEQ ID NO: 9 - DNA, 19 bp, synthetic construct
  source 1..19: mol_type = other DNA
gtgtggacaagatccggaa

SEQ ID NO: 10 - DNA, 19 bp, synthetic construct
  source 1..19: mol_type = other DNA
cgagaagactggaagtcca

SEQ ID NO: 11 - DNA, 19 bp, synthetic construct
  source 1..19: mol_type = other DNA
ccctgtacttcatcgacaa

SEQ ID NO: 12 - DNA, 19 bp, synthetic construct
  source 1..19: mol_type = other DNA
gcacatcaatctacaccca

SEQ ID NO: 13 - DNA, 19 bp, synthetic construct
  source 1..19: mol_type = other DNA
aggatgcaaagacgaagaa

SEQ ID NO: 14 - DNA, 19 bp, synthetic construct
  source 1..19: mol_type = other DNA
ggtttgggaaagatggata

SEQ ID NO: 15 - DNA, 19 bp, synthetic construct
  source 1..19: mol_type = other DNA
cgattatattggtgcgttt

SEQ ID NO: 16 - RNA, 21 nt, synthetic construct
  source 1..21: mol_type = other RNA
  modified_base 20..21: mod_base = OTHER; note = thymine
caacgattcccagatcgaatt

SEQ ID NO: 17 - RNA, 21 nt, synthetic construct
  source 1..21: mol_type = other RNA
  modified_base 20..21: mod_base = OTHER; note = thymine
cctcctaaagaggatataatt

SEQ ID NO: 18 - RNA, 21 nt, synthetic construct
  source 1..21: mol_type = other RNA
  modified_base 20..21: mod_base = OTHER; note = thymine
ggaaattcctagaacataatt

SEQ ID NO: 19 - RNA, 21 nt, synthetic construct
  source 1..21: mol_type = other RNA
  modified_base 20..21: mod_base = OTHER; note = thymine
ggattctgtattatggatatt

SEQ ID NO: 20 - RNA, 21 nt, synthetic construct
  source 1..21: mol_type = other RNA
  modified_base 20..21: mod_base = OTHER; note = thymine
cacaaagagaggattgctatt

SEQ ID NO: 21 - RNA, 21 nt, synthetic construct
  source 1..21: mol_type = other RNA
  modified_base 20..21: mod_base = OTHER; note = thymine
ggcatgctgaagagacgaatt

SEQ ID NO: 22 - RNA, 21 nt, synthetic construct
  source 1..21: mol_type = other RNA
  modified_base 20..21: mod_base = OTHER; note = thymine
cggcatgataacaaggttatt

SEQ ID NO: 23 - RNA, 21 nt, synthetic construct
  source 1..21: mol_type = other RNA
  modified_base 20..21: mod_base = OTHER; note = thymine
gcttaaccctagttcacgatt

SEQ ID NO: 24 - RNA, 21 nt, synthetic construct
  source 1..21: mol_type = other RNA
  modified_base 20..21: mod_base = OTHER; note = thymine
gtgtggacaagatccggaatt

SEQ ID NO: 25 - RNA, 21 nt, synthetic construct
  source 1..21: mol_type = other RNA
  modified_base 20..21: mod_base = OTHER; note = thymine
cgagaagactggaagtccatt

SEQ ID NO: 26 - RNA, 21 nt, synthetic construct
  source 1..21: mol_type = other RNA
  modified_base 20..21: mod_base = OTHER; note = thymine
ccctgtacttcatcgacaatt

SEQ ID NO: 27 - RNA, 21 nt, synthetic construct
  source 1..21: mol_type = other RNA
  modified_base 20..21: mod_base = OTHER; note = thymine
gcacatcaatctacacccatt

SEQ ID NO: 28 - RNA, 21 nt, synthetic construct
  source 1..21: mol_type = other RNA
  modified_base 20..21: mod_base = OTHER; note = thymine
aggatgcaaagacgaagaatt

SEQ ID NO: 29 - RNA, 21 nt, synthetic construct
  source 1..21: mol_type = other RNA
  modified_base 20..21: mod_base = OTHER; note = thymine
ggtttgggaaagatggatatt

SEQ ID NO: 30 - RNA, 21 nt, synthetic construct
  source 1..21: mol_type = other RNA
  modified_base 20..21: mod_base = OTHER; note = thymine
cgattatattggtgcgttttt

SEQ ID NO: 31 - RNA, 21 nt, synthetic construct
  source 1..21: mol_type = other RNA
  modified_base 20..21: mod_base = OTHER; note = thymine
ttcgatctgggaatcgttgtt

SEQ ID NO: 32 - RNA, 21 nt, synthetic construct
  source 1..21: mol_type = other RNA
  modified_base 20..21: mod_base = OTHER; note = thymine
ttatatcctctttaggaggtt

SEQ ID NO: 33 - RNA, 21 nt, synthetic construct
  source 1..21: mol_type = other RNA
  modified_base 20..21: mod_base = OTHER; note = thymine
ttatgttctaggaatttcctt

SEQ ID NO: 34 - RNA, 21 nt, synthetic construct
  source 1..21: mol_type = other RNA
  modified_base 20..21: mod_base = OTHER; note = thymine
tatccataatacagaatcctt

SEQ ID NO: 35 - RNA, 21 nt, synthetic construct
  source 1..21: mol_type = other RNA
  modified_base 20..21: mod_base = OTHER; note = thymine
tagcaatcctctctttgtgtt

SEQ ID NO: 36 - RNA, 21 nt, synthetic construct
  source 1..21: mol_type = other RNA
  modified_base 20..21: mod_base = OTHER; note = thymine
ttcgtctcttcagcatgcctt

SEQ ID NO: 37 - RNA, 21 nt, synthetic construct
  source 1..21: mol_type = other RNA
  modified_base 20..21: mod_base = OTHER; note = thymine
taaccttgttatcatgccgtt

SEQ ID NO: 38 - RNA, 21 nt, synthetic construct
  source 1..21: mol_type = other RNA
  modified_base 20..21: mod_base = OTHER; note = thymine
tcgtgaactagggttaagctt

SEQ ID NO: 39 - RNA, 21 nt, synthetic construct
  source 1..21: mol_type = other RNA
  modified_base 20..21: mod_base = OTHER; note = thymine
ttccggatcttgtccacactt

SEQ ID NO: 40 - RNA, 21 nt, synthetic construct
  source 1..21: mol_type = other RNA
  modified_base 20..21: mod_base = OTHER; note = thymine
tggacttccagtcttctcgtt

SEQ ID NO: 41 - RNA, 21 nt, synthetic construct
  source 1..21: mol_type = other RNA
  modified_base 20..21: mod_base = OTHER; note = thymine
ttgtcgatgaagtacagggtt

SEQ ID NO: 42 - RNA, 21 nt, synthetic construct
  source 1..21: mol_type = other RNA
  modified_base 20..21: mod_base = OTHER; note = thymine
tgggtgtagattgatgtgctt

SEQ ID NO: 43 - RNA, 21 nt, synthetic construct
  source 1..21: mol_type = other RNA
  modified_base 20..21: mod_base = OTHER; note = thymine
ttcttcgtctttgcatccttt

SEQ ID NO: 44 - RNA, 21 nt, synthetic construct
  source 1..21: mol_type = other RNA
  modified_base 20..21: mod_base = OTHER; note = thymine
tatccatctttcccaaacctt

SEQ ID NO: 45 - RNA, 21 nt, synthetic construct
  source 1..21: mol_type = other RNA
  modified_base 20..21: mod_base = OTHER; note = thymine
aaacgcaccaatataatcgtt

SEQ ID NO: 46 - DNA, 19 bp, synthetic construct
  source 1..19: mol_type = other DNA
ttctccgaacgtgtcacgt

SEQ ID NO: 47 - DNA, 20 bp, synthetic construct
  source 1..20: mol_type = other DNA
tctgacttcaacagcgacac

SEQ ID NO: 48 - DNA, 20 bp, synthetic construct
  source 1..20: mol_type = other DNA
gccaaattcgttgtcatacc

SEQ ID NO: 49 - DNA, 21 bp, synthetic construct
  source 1..21: mol_type = other DNA
ctgtagccctgtacttcatcg

SEQ ID NO: 50 - DNA, 23 bp, synthetic construct
  source 1..23: mol_type = other DNA
ctaccacatattcctgaccatcc

SEQ ID NO: 51 - RNA, 21 nt, synthetic construct
  source 1..21: mol_type = other RNA
  modified_base 20..21: mod_base = OTHER; note = thymine
  modified_base 2: mod_base = OTHER; note = 2-O-methyl
  modified_base 4: mod_base = OTHER; note = 2-O-methyl
  modified_base 8: mod_base = OTHER; note = 2-O-methyl
  modified_base 13..15: mod_base = OTHER; note = 2-O-methyl
gtgtggacaagatccggaatt

SEQ ID NO: 52 - RNA, 21 nt, synthetic construct
  source 1..21: mol_type = other RNA
  modified_base 20..21: mod_base = OTHER; note = thymine
  modified_base 1..4: mod_base = OTHER; note = 2-O-methyl
  modified_base 8..11: mod_base = OTHER; note = 2-O-methyl
  modified_base 13..15: mod_base = OTHER; note = 2-O-methyl
  modified_base 17: mod_base = OTHER; note = 2-O-methyl
  modified_base 19: mod_base = OTHER; note = 2-O-methyl
ttccggatcttgtccacactt

SEQ ID NO: 53 - RNA, 21 nt, synthetic construct
  source 1..21: mol_type = other RNA
  modified_base 20..21: mod_base = OTHER; note = thymine
  modified_base 1..9: mod_base = OTHER; note = 2-O-methyl
  modified_base 10..12: mod_base = OTHER; note = 2-deoxy-2-fluoro
  modified_base 13..19: mod_base = OTHER; note = 2-O-methyl
  modified_base 1^2: mod_base = OTHER; note = Phosphorothioate
  modified_base 2^3: mod_base = OTHER; note = Phosphorothioate
gtgtggacaagatccggaatt

SEQ ID NO: 54 - RNA, 21 nt, synthetic construct
  source 1..21: mol_type = other RNA
  modified_base 20..21: mod_base = OTHER; note = thymine
  modified_base 1..19: mod_base = OTHER; note = 2-O-methyl
  modified_base 1^2: mod_base = OTHER; note = Phosphorothioate
  modified_base 2^3: mod_base = OTHER; note = Phosphorothioate
  modified_base 17^18: mod_base = OTHER; note = Phosphorothioate
  modified_base 18^19: mod_base = OTHER; note = Phosphorothioate
ttccggatcttgtccacactt

SEQ ID NO: 55 - RNA, 21 nt, synthetic construct
  source 1..21: mol_type = other RNA
  modified_base 20..21: mod_base = OTHER; note = thymine
  modified_base 2: mod_base = p
  modified_base 4: mod_base = p
  modified_base 13: mod_base = p
gtgtggacaagatccggaatt

SEQ ID NO: 56 - RNA, 21 nt, synthetic construct
  source 1..21: mol_type = other RNA
  modified_base 20..21: mod_base = OTHER; note = thymine
  modified_base 1..2: mod_base = p
  modified_base 8: mod_base = p
  modified_base 10..11: mod_base = p
  modified_base 13: mod_base = p
ttccggatcttgtccacactt

(19) United States
(12) Patent Application Publication
(10) Pub. No.: US 2026/0139254 A1
(43) Pub. Date: May 21, 2026

(54) INTERFERING RNA FOR INHIBITING TOP1 GENE EXPRESSION AND USE THEREOF

(71) Applicant: JENKEM TECHNOLOGY CO., LTD. (BEIJING), Beijing (CN)

(72) Inventors: Mai YANG, Beijing (CN); Meina Lin, Beijing (CN); Hongli JIA, Beijing (CN); Xiaomeng CHEN, Beijing (CN); Jun GUO, Beijing (CN); Qingbin WANG, Beijing (CN); Xuan ZHAO, Beijing (CN)

(21) Appl. No.: 19/114,761

(22) PCT Filed: Sep. 22, 2023
(86) PCT No.: PCT/CN2023/120758

§ 371 Date: Jun. 5, 2025

(30) Foreign Application Priority Data
Sep. 23, 2022 (CN) 202211167614.X

(51) Int. Cl. C12N 15/113; C12N 15/88

(52) U.S. Cl. CPC C12N 15/1137; C12N 15/88; C12N 2310/14; C12N 2310/315; C12N 2310/321

(57) ABSTRACT

The present invention provides an interfering RNA targeting TOP1 gene. The interfering RNA can reduce the expression of TOP1, thereby treating a disease related to TOP1 expression. The present invention further provides a lipid nanoparticle drug comprising the interfering RNA for treating a disease related to TOP1 expression. The present invention further provides an antibody-nucleic acid conjugate drug comprising the interfering RNA for treating a disease related to TOP1 expression.

### CROSS-REFERENCE TO RELATED APPLICATIONS

This application is the U.S. national phase of International Application No. PCT/CN2023/120758 filed Sep. 22, 2023, which designated the U.S. and claims priority to CN 202211167614.X filed Sep. 23, 2022, the entire contents of each of which are hereby incorporated by reference.

### REFERENCE TO SEQUENCE LISTING SUBMITTED ELECTRONICALLY

The content of the electronically submitted sequence listing (Name: 8575-0086_SEQUENCE_LISTING.xml; Size: 82,468 bytes; and Date of Creation: Jun. 15, 2025) filed with the application is incorporated herein by reference in its entirety.

### TECHNICAL FIELD

The present invention relates to the technical field of molecular biology and biomedicine, and in particular to an interfering RNA, particularly siRNA, for inhibiting TOP1 expression and use thereof.

### BACKGROUND

Topoisomerases are essential enzymes in higher eukaryotic cells, with Topoisomerase I (TOP1) belongs to a type I topoisomerase. TOP1 forms a TOP1-DNA cleavage complex (TOP1cc) with DNA, and catalyzing transient single-strand breaks in DNA without ATP hydrolysis for energy. The cleavage and religation effects of TOP1 on the phosphate backbone can facilitate changing the topologic structure of DNA and maintaining the topologic stability during various processes including DNA replication, transcription, repair, recombination, and the like.

TOP1 is one of the targets of drugs in treating various tumors, and currently, TOP1-targeted drugs are divided into two categories: camptothecin drugs, such as topotecan, irinotecan, and belotecan; and ADC drugs carrying camptothecin small molecules. They share essentially the same mechanism of action: camptothecin is capable of binding to TOP1cc reversely to disrupt the replication or transcription process that should have been smooth during DNA replication or transcription, thus causing DNA damage. However, camptothecin drugs have their own limitations: 1) a relatively long period is needed for the binding of camptothecin to TOP1cc to cause DNA damage; 2) camptothecin brings certain side effects, e.g., leukopenia, diarrhea, etc., limiting its dose of use; and 3) the structural alteration of camptothecin renders it unable to target TOP1, but able to bind to serum albumin. In order to solve these problems, the development of camptothecin derivatives and non-camptothecin drugs is of great significance.

Studies have shown that the colon cancer and breast cancer cell lines with stably knocked-down TOP1 had a TOP1 expression of 10%-20% of the normal level and all exhibited phenomena such as chromosomal aberration, replication defects, altered gene expression levels, etc. Small interfering RNA (siRNA) is an RNA fragment of about 20 nt in length, and can participate in the formation of an RNA-induced silencing complex (RISC) and target and degrade a specific mRNA by means of base complementary pairing in vivo. Therefore, the interfering RNA is used to silence the TOP1 expression level in the present application.

### SUMMARY

In a first aspect of the present invention, provided is an interfering RNA targeting TOP1 gene. The interfering RNA inhibits TOP1 expression.

A target site sequence of the interfering RNA comprises a nucleotide sequence set forth in any one or two or more of SEQ ID NOs: 1-15. Preferably, the target site sequence of the interfering RNA is set forth in any one of the nucleotide sequences of SEQ ID NOs: 1-15. Further preferably, the target site sequence of the interfering RNA comprises the nucleotide sequence set forth in any one or two or more of SEQ ID NO: 1, 9, or 10.

The interfering RNA comprises one or a combination of two or more of an siRNA, a dsRNA, an shRNA, an aiRNA, or an miRNA.

In one specific embodiment of the present invention, the interfering RNA is an siRNA.

The interfering RNA is 17-25 nt, preferably 19-21 nt, e.g., 17, 18, 19, 20, 21, 22, 23, 24, or 25 nt, in length.

The interfering RNA further comprises an over-hang to increase the stability and activity of the interfering RNA.

Preferably, the interfering RNA comprises 1-10 over-hangs, further preferably, 2-4 over-hangs, e.g., 1, 2, 3, 4, 5, 6, 7, 8, 9, or 10 over-hangs.

The over-hang is located at the 3′ terminus of a sense strand and/or an antisense strand of the interfering RNA.

The over-hang is a deoxynucleoside; preferably, the over-hang may be n identical or different deoxynucleosides (e.g., deoxythymidine (dT), deoxycytidine (dC), deoxyuridine (dU), etc.), with n being an integer of 1-10 (e.g., 1, 2, 3, 4, 5, 6, 7, 8, 9, or 10).

In one specific embodiment of the present invention, the terminus of the sense strand and/or the antisense strand of the interfering RNA has 2 identical over-hangs.

In one specific embodiment of the present invention, the terminus of the sense strand and/or the antisense strand of the interfering RNA has 2 different over-hangs.

In one specific embodiment of the present invention, the over-hang is dTdT, dTdC, or dUdU.

In one specific embodiment of the present invention, the interfering RNA comprises a sense strand and/or an antisense strand.

Preferably, the sense strand and the antisense strand are complementarily paired.

Preferably, the sense strand comprises a nucleotide sequence set forth in any one or two or more of SEQ ID NOs: 16-30. Further preferably, the sense strand is set forth in any one of the nucleotide sequences of SEQ ID NOs: 16-30. Further preferably, the sense strand comprises the nucleotide sequence set forth in any one or two or more of SEQ ID NO: 16, 24, or 25.

Preferably, the antisense strand comprises a nucleotide sequence set forth in any one or two or more of SEQ ID NOs: 31-45. Further preferably, the antisense strand is set forth in any one of the nucleotide sequences of SEQ ID NOs: 31-45. Further preferably, the antisense strand comprises the nucleotide sequence set forth in any one or two or more of SEQ ID NO: 31, 39, or 40.

In one specific embodiment of the present invention, the interfering RNA may further comprise at least one modification. The modified interfering RNA has better properties, e.g., higher stability, lower immune stimulation, etc., compared to a corresponding unmodified interfering RNA.

The modification comprises modifications on chemical structures of a base, a sugar ring, and/or a phosphate.

Preferably, the modification on a base includes, but is not limited to, a pyrimidine modification at position 5, a purine modification at position 8, and/or a 5-bromouracil substitution.

Preferably, the modification on a sugar ring includes, but is not limited to, a substitution of 2′-OH with H, OZ, Z, halo, SH, SZ, NH₂, NHZ, NZ₂, or CN group, wherein Z is an alkyl group.

The alkyl group refers to linear or branched hydrocarbyl that does not contain unsaturated bonds, and the hydrocarbyl is linked to the rest of the molecule via a single bond. A typical alkyl group contains 1-20 (e.g., 1, 2, 3, 4, 5, 6, 7, 8, 9, 10, 11, 12, 13, 14, 15, 16, 17, 18, 19, or 20) carbon atoms, e.g., methyl, ethyl, n-propyl, isopropyl, n-butyl, sec-butyl, isobutyl, tert-butyl, n-pentyl, isopentyl, neopentyl, tert-pentyl, n-hexyl, isohexyl, n-heptyl, isoheptyl, n-octyl, nonyl, decyl, undecyl, 1-methylundecyl, dodecyl, tridecyl, tetradecyl, pentadecyl, hexadecyl, heptadecyl, octadecyl, nonadecyl, eicosyl, etc.

Preferably, the modification on a phosphate backbone includes, but is not limited to, a phosphorothioate modification.

Preferably, the modification further comprises nucleotides having inosine, queuosine, xanthine, 2′-methylribose, a non-natural phosphodiester bond (e.g., methylphosphonate and phosphorothioate), and/or a peptide.

In one specific embodiment of the present invention, the modification may be methylation, fluorination, a phosphorothioate modification, a pseudouridine modification, etc.

The modification may occur at any site in the sequence, e.g., at some sites or all sites.

The same sequence may have an identical or different modification at some sites or all sites.

In one specific embodiment of the present invention, the interfering RNA comprises one or a combination of two or more of the following combinations of the target site sequence and the sense strand and antisense strand sequences:
A) SEQ ID NO: 1, SEQ ID NO: 16, and SEQ ID NO: 31;
B) SEQ ID NO: 2, SEQ ID NO: 17, and SEQ ID NO: 32;
C) SEQ ID NO: 3, SEQ ID NO: 18, and SEQ ID NO: 33;
D) SEQ ID NO: 4, SEQ ID NO: 19, and SEQ ID NO: 34;
E) SEQ ID NO: 5, SEQ ID NO: 20, and SEQ ID NO: 35;
F) SEQ ID NO: 6, SEQ ID NO: 21, and SEQ ID NO: 36;
G) SEQ ID NO: 7, SEQ ID NO: 22, and SEQ ID NO: 37;
H) SEQ ID NO: 8, SEQ ID NO: 23, and SEQ ID NO: 38;
I) SEQ ID NO: 9, SEQ ID NO: 24, and SEQ ID NO: 39;
J) SEQ ID NO: 10, SEQ ID NO: 25, and SEQ ID NO: 40;
K) SEQ ID NO: 11, SEQ ID NO: 26, and SEQ ID NO: 41;
L) SEQ ID NO: 12, SEQ ID NO: 27, and SEQ ID NO: 42;
M) SEQ ID NO: 13, SEQ ID NO: 28, and SEQ ID NO: 43;
N) SEQ ID NO: 14, SEQ ID NO: 29, and SEQ ID NO: 44;
O) SEQ ID NO: 15, SEQ ID NO: 30, and SEQ ID NO: 45;
P) SEQ ID NO: 9, SEQ ID NO: 51, and SEQ ID NO: 52;
Q) SEQ ID NO: 9, SEQ ID NO: 53, and SEQ ID NO: 54; and
R) SEQ ID NO: 9, SEQ ID NO: 55, and SEQ ID NO: 56.

Preferably, the interfering RNA comprises combination A), combination I), and/or combination J).

In one specific embodiment of the present invention, the interfering RNA comprises one or a combination of two or more of the nucleic acid sequences shown in Table 1 or Table 13.

The interfering RNA can be prepared and obtained by any method in the prior art, e.g., chemical synthesis, etc.

In a second aspect of the present invention, provided is a delivery system comprising the interfering RNA described above.

Preferably, the delivery system further comprises a vector.

Preferably, the vector may be any vector suitable for delivering the interfering RNA of the present invention described above to a target tissue or a target cell, such as those disclosed in the prior art (e.g., Chen Zhonghua, Zhu Desheng, Li Jun, Huang Zhanqin, “Advances in non-viral vector research of siRNA”. Chinese Pharmacological Bulletin, 2015, 31 (7): 910-4; Wang Rui, Qu Bingnan, Yang Jing, “Advances in research of siRNA-carrying nano preparation”, China Pharmacy 2017, 28 (31): 4452-4455).

In one specific embodiment of the present invention, the vector is a viral vector; preferably, the viral vector includes, but is not limited to, one or two or more of a lentivirus vector, a retrovirus vector, an adenovirus vector, an adeno-associated virus vector, a poxvirus vector, a herpesvirus vector, etc.

In one specific embodiment of the present invention, the vector is a non-viral vector; preferably, the non-viral vector includes, but is not limited to, any one or a combination of two or more of a liposome, a lipid nanoparticle (LNP), a polymer, a polypeptide, an antibody, an aptamer, or N-acetylgalactosamine (GalNAc); further preferably, the non-viral vector comprises a lipid nanoparticle (LNP). The weight ratio of the interfering RNA to the non-viral vector may be any value in the range of 1:1 to 1:50, preferably any value in the range of 1:1 to 1:10 (e.g., 1:1, 1:5, 1:6, 1:7, 1:8, 1:9, 1:10, 1:15, 1:20, 1:25, 1:30, 1:35, 1:40, 1:45, or 1:50).

Preferably, the lipid nanoparticle/liposome described above comprises one or a combination of two or more of a cationic lipid, a neutral lipid, a polyethylene glycol lipid, a steroidal lipid, or an anionic lipid.

Further preferably, the cationic lipid comprises one or a combination of two or more of stearamide (SA), lauryltrimethylammonium bromide, hexadecyltrimethylammonium bromide, myristyltrimethylammonium bromide, dimethyldioctadecylammonium bromide (DDAB), [(4-hydroxybutyl)azanediyl]di(hexane-6,1-diyl) bis(2-hexyldecanoate) (ALC-0315), 1,2-dioleoyloxy-3-(trimethylammonium)propane (DOTAP), 1,2-di-(9Z-octadecenoyl)-3-trimethylammonium-propane and 1,2-dihexadecanoyl-3-trimethylammonium-propane, 3β-[N—(N,N-dimethylaminoethane)-carbamoyl]cholesterol (DC-cholesterol), dimethyldioctadecylammonium (DDA), 1,2-dimyristoyl-3-trimethylammonium propane (DMTAP), dipalmitoyl(C16:0)trimethylammonium propane (DPTAP), distearoyltrimethylammonium propane (DSTAP), N-[1-(2,3-diallyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium propane (DODAP), 1,2-dilinoleyloxy-3-dimethylaminopropane (DLinDMA), 1,2-ditetradecanoyl-3-dimethylammonium-propane, 1,2-dihexadecanoyl-3-dimethylammonium-propane and 1,2-dioctadecanoyl-3-dimethylammonium-propane, 1,2-dioleoyl-c-(4′-trimethylammonium)-butanoyl-sn-glycerol (DOTB), dioctadecylamidoalanyl spermine, SAINT-2, polycationic lipid 2,3-dioleoyloxy-N-[2(spermine-carboxamido)ethyl]-N,N-dimethyl-1-propanaminium trifluoroacetate (DOSPA),

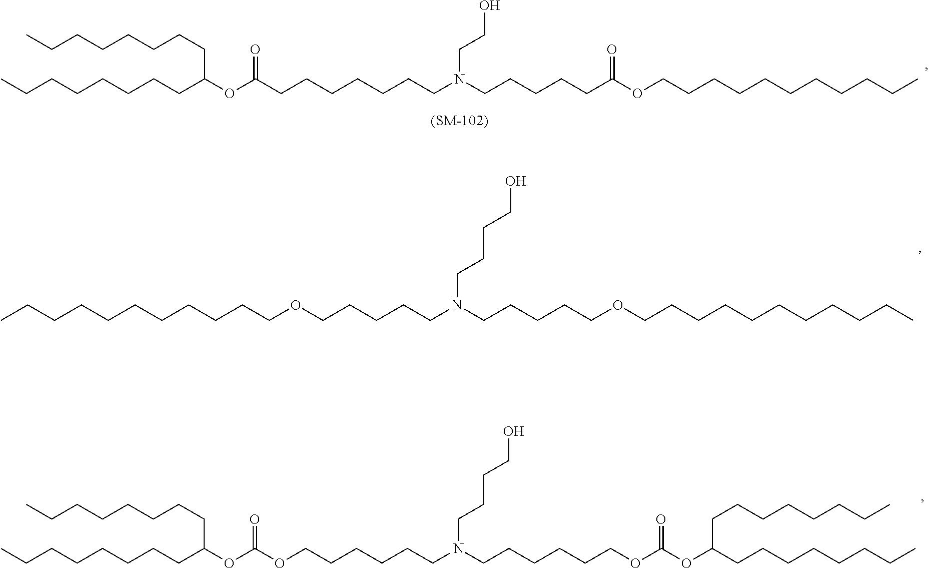

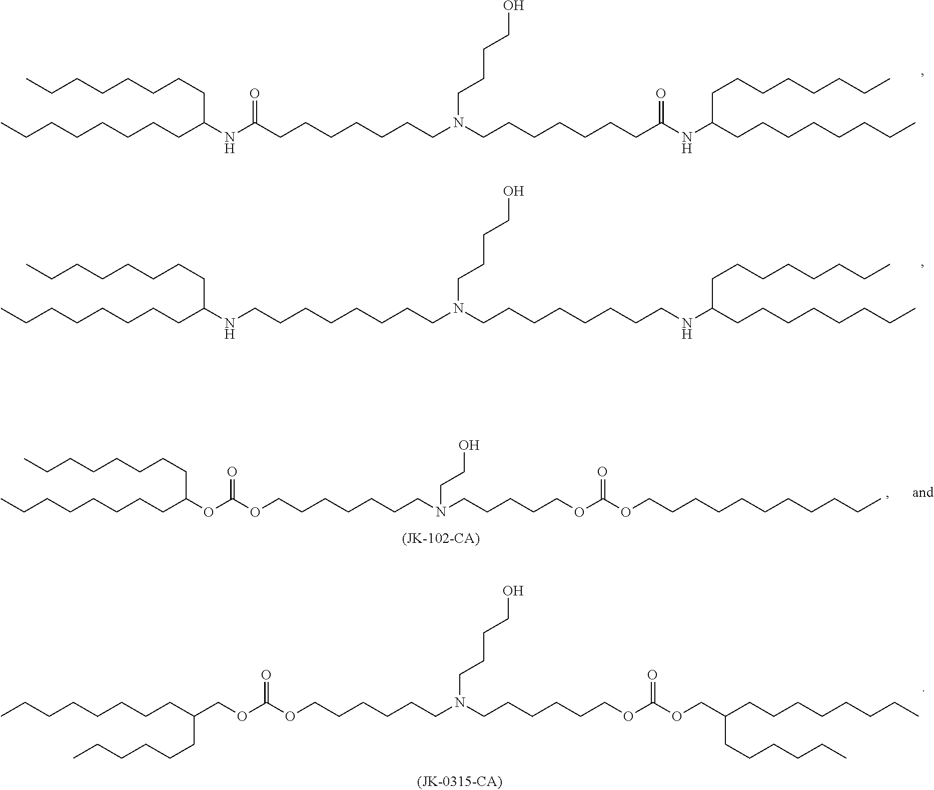

Preferably, the cationic lipid is a steroid-cationic lipid compound,

having a structure of one or two or more of the following:

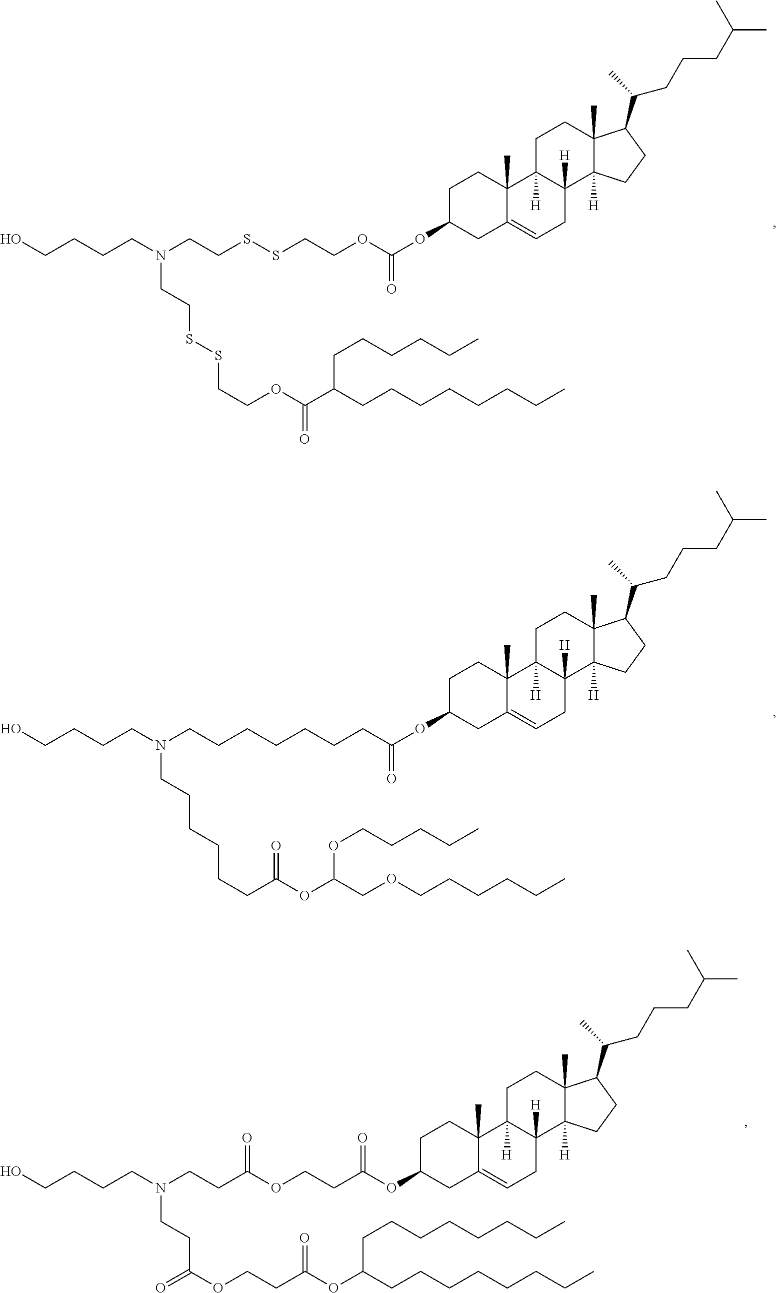

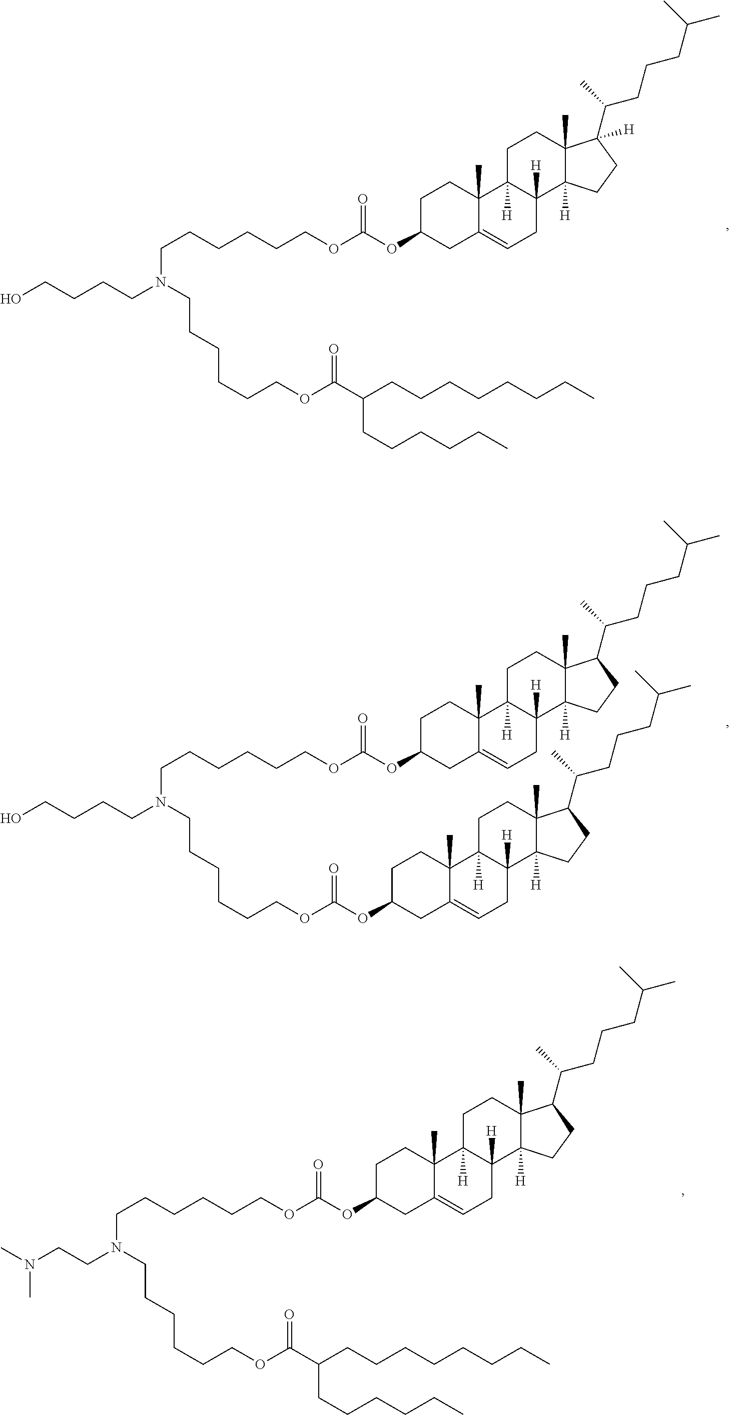

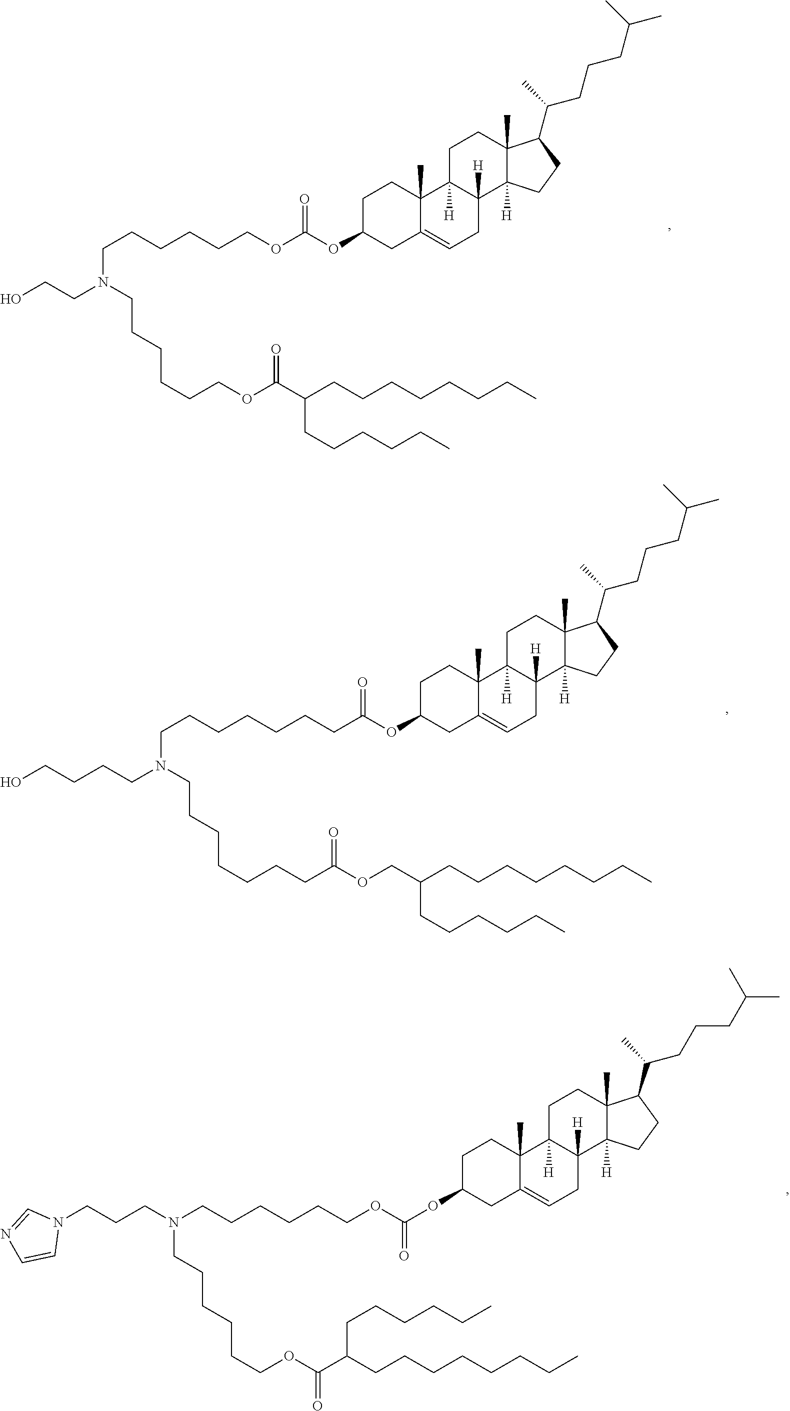

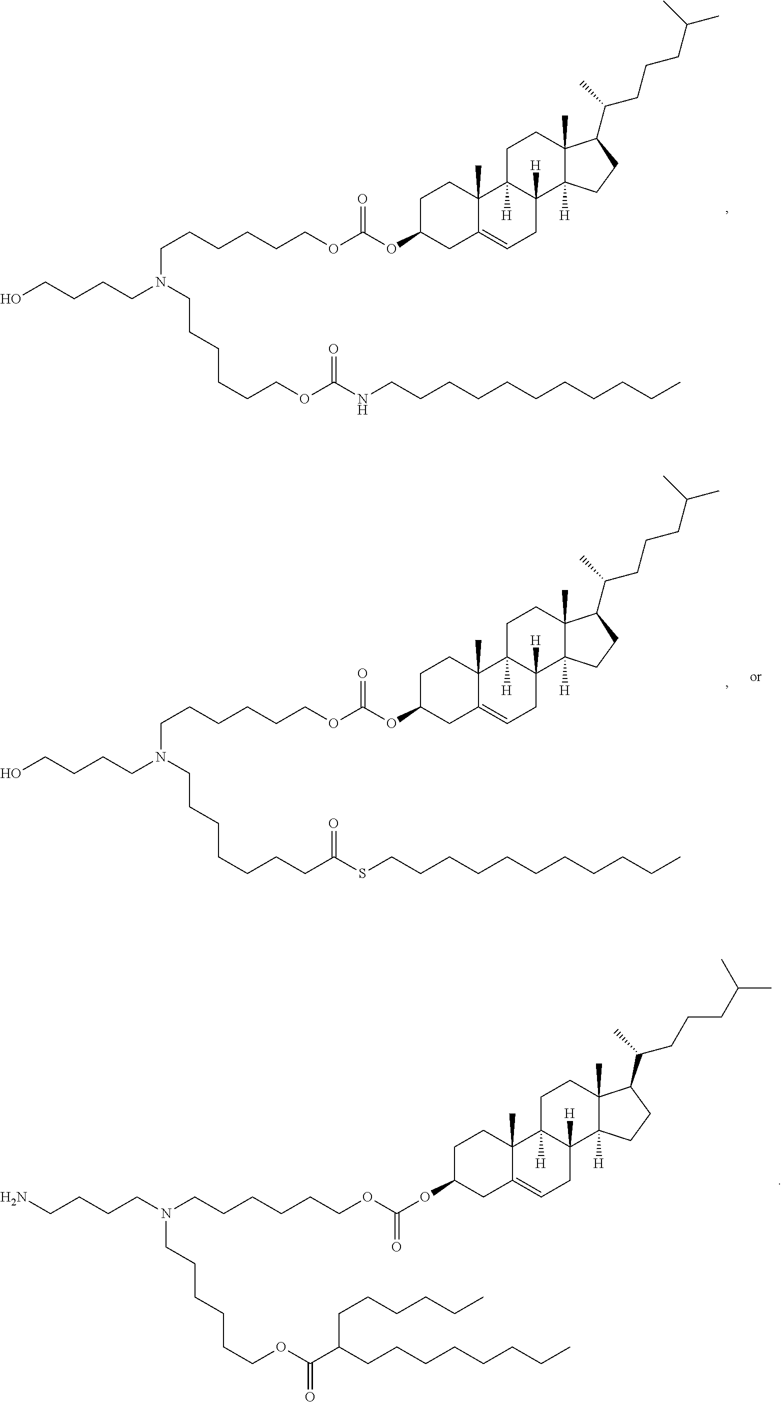

Further preferably, the neutral lipid comprises one or a combination of two or more of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), or distearoylphosphatidylethanolamine (DSPE).

Further preferably, the polyethylene glycol lipid comprises one or a combination of two or more of 2-[(polyethylene glycol)-2000]-N,N-ditetradecylacetamide (ALC-0159), 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disterol glycerol (PEG-DSG), PEG-dipalmitoyl, PEG-dioleyl, PEG-distearyl, PEG-diacylglycerol amide (PEG-DAG), PEG-dipalmitoyl phosphatidylethanolamine (PEG-DPPE) or PEG-1,2-dimyristoyloxypropyl-3-amine (PEG-c-DMA),

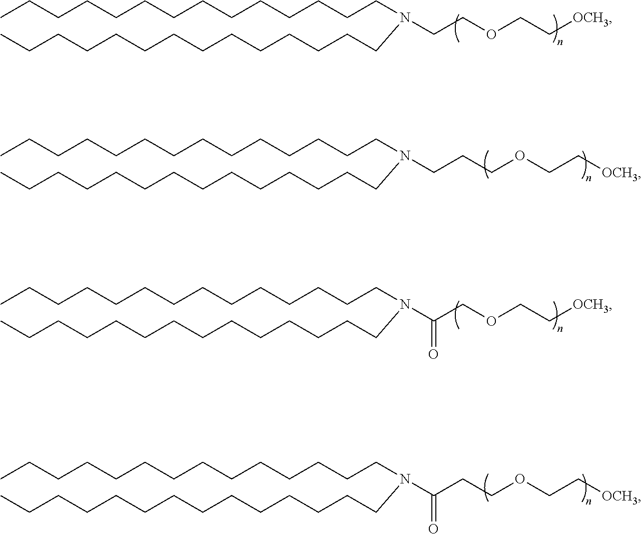

etc.,

wherein n is selected from integers of 20-300, e.g., 20, 30, 50, 80, 100, 150, 200, 250, 300, etc. Preferably, the polyethylene glycol lipid is a polyethylene glycol lipid with a single molecular weight, and preferably, the polyethylene glycol lipid comprises:

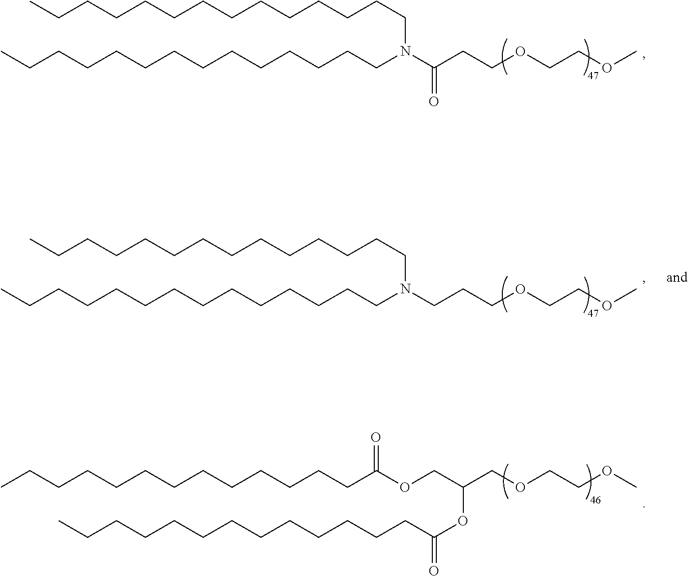

Further preferably, the anionic liposome comprises: dioleoyl phosphatidylglycerol and/or dioleoyl phosphatidylethanolamine, etc.

Further preferably, the steroidal lipid comprises one or a combination of two or more of avenasterol, β-sitosterol, brassicasterol, ergocalciferol, campesterol, cholestanol, cholesterol, coprostanol, dehydrocholesterol, desmosterol, dihydroergocalciferol, dihydrocholesterol, dihydroergosterol, dinosterol, epicholesterol, ergosterol, fucosterol, hexahydrolumisterol, hydroxycholesterol, lanosterol, lumisterol, saringosterol, sitostanol, sitosterol, stigmastanol, stigmasterol, cholic acid, glycocholic acid, taurocholic acid, deoxycholic acid, or lithocholic acid. Preferably, the polymer described above may be a synthetic polymer (e.g., polyethyleneimine, cyclodextrin, etc.) or a natural polymer (e.g., chitosan, atelocollagen, etc.) or a mixture thereof. Preferably, the polypeptide described above may be a cell penetrating peptide (CPP) (e.g., protamine, Tat peptide, transportan peptide, penetratin peptide, oligo-arginine peptide, etc.). Preferably, the antibody described above may be a single chain antibody (e.g., scFv-tp, scFv-9R, etc.).

The delivery system of the present application can also encapsulate various components beneficial to the human body, and directly deliver the components into cells to exert their effects, thereby achieving the desired results more quickly and effectively.

In a third aspect of the present invention, provided is a cell comprising the interfering RNA described above or the delivery system described above.

The expression level of TOP1 in the cell is inhibited.

The cell may be a tumor cell.

In a fourth aspect of the present invention, provided is a preparation method for a cell, comprising introducing into a cell the interfering RNA described above or the delivery system described above.

In a fifth aspect of the present invention, provided is a lipid nanoparticle comprising the interfering RNA described above.

Preferably, the lipid nanoparticle further comprises one or two or more of a polyethylene glycol lipid compound, a cationic lipid, a steroidal lipid, or a neutral lipid.

The polyethylene glycol lipid compound, the cationic lipid, the steroidal lipid, and the neutral lipid are as defined in the second aspect of the present application.

In one specific embodiment of the present invention, the lipid nanoparticle comprises the interfering RNA described above, as well as a polyethylene glycol lipid compound, a cationic lipid (excluding a steroid-cationic lipid compound), a steroidal lipid, and a neutral lipid.

Preferably, in the lipid nanoparticle, the polyethylene glycol lipid compound, the cationic lipid, the steroidal lipid, and the neutral lipid are in a molar ratio of (0.5-5):(30-55):(30-55):(5-20), such as (0.5, 1, 2, 3, 4, or 5):(30, 31, 32, 33, 34, 35, 36, 37, 38, 39, 40, 41, 42, 43, 44, 45, 46, 47, 48, 49, 50, 51, 52, 53, 54, or 55):(30, 31, 32, 33, 34, 35, 36, 37, 38, 39, 40, 41, 42, 43, 44, 45, 46, 47, 48, 49, 50, 51, 52, 53, 54, or 55):(5, 6, 7, 8, 9, 10, 11, 12, 13, 14, 15, 16, 17, 18, 19, or 20). Preferably, the polyethylene glycol lipid compound, the cationic lipid, the steroidal lipid, and the neutral lipid are in a molar ratio of (1-5):(35-50):(40-50):(8-15).

In one specific embodiment of the present invention, the lipid nanoparticle comprises the interfering RNA described above, as well as a steroid-cationic lipid compound, a second lipid, and a polyethylene glycol lipid. Among them, the second lipid is selected from a neutral lipid, a zwitterionic lipid, and an anionic lipid.

Preferably, in the lipid nanoparticle, the steroid-cationic lipid, the second lipid, and the polyethylene glycol lipid are in a molar ratio of (10-30):(60-80):(10-25), such as (10, 15, 20, 25, or 30):(60, 65, 70, 75, or 80):(10, 15, 20, or 25), preferably (20-30):(60-70):(10-20).

The lipid nanoparticle of the present application can also encapsulate various components beneficial to the human body, and directly deliver the components into cells to exert their effects, thereby achieving the desired results more quickly and effectively.

The lipid nanoparticle can be prepared by using a conventional method for preparing a lipid nanoparticle in the art, e.g., high pressure homogenization, emulsion precipitation, ultrasonic dispersion, etc.

In a sixth aspect of the present invention, provided is a medicament or a kit, comprising the interfering RNA described above, the delivery system described above, the lipid nanoparticle described above, or the cell described above.

The medicament further comprises a pharmaceutically acceptable auxiliary material.

Preferably, the pharmaceutically acceptable auxiliary material includes, but is not limited to, a carrier, a diluent, a binder, a lubricant, a wetting agent, etc.

Preferably, the administration mode of the medicament includes, but is not limited to, oral administration, intestinal administration, subcutaneous injection, intramuscular injection, intravenous injection, nasal administration, transdermal administration, subconjunctival administration, intraocular administration, orbital administration, retrobulbar administration, retinal administration, choroidal administration, intrathecal injection, etc.

Preferably, the dosage form of the medicament includes, but is not limited to, a tablet, a capsule, a pill, an injection, an inhalant, a troche, a suppository, an emulsion, a microemulsion, a submicroemulsion, a nanoparticle, a gel, a powder, a suspoemulsion, a cream, a jelly, a spray, etc. The various dosage forms of the medicament can be prepared according to conventional production methods in the field of pharmaceuticals.

Preferably, the mass content of the interfering RNA described above, the delivery system described above, or the cell described above in the medicament may be 1%-100%, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, or 100%.

In a seventh aspect of the present invention, provided is an antibody-nucleic acid drug conjugate, comprising the interfering RNA described above or the delivery system described above or the lipid nanoparticle described above.

The antibody-nucleic acid drug conjugate comprises one or more interfering RNAs, as well as an antibody.

In the antibody-nucleic acid drug conjugate, the antibody and the nucleic acid may be directly linked or linked via a linking group or a linker peptide.

The antibody-nucleic acid drug conjugate has a general formula (I):

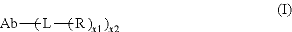

wherein R is the interfering RNA described above;
x1 is an integer of 1-144; preferably, x1 is an integer of 1-9; more preferably, x1 is an integer of 1-3;
x2 is an integer of 1-8; preferably, x2 is an integer of 1-2;
Ab is an antibody, a protein, or a polypeptide;
L is a linking unit connecting Ab and R.

The moiety L has a structure of general formula (II):

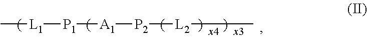

wherein
x3 in formula II is selected from integers of 1-12, preferably 1-3;
x4 in formula II is selected from integers of 1-12, preferably 1-3;
P₁ and P₂ are identical or different polyethylene glycol residues;
L₁ is a linking unit connecting Ab and P₁;
L₂ is a linking unit connecting P₂ and R;
A₁ is a linking unit connecting P₁ and P₂;
preferably, P₁ and P₂ are independently selected from linear, Y-shaped, and multi-branched polyethylene glycol residues;
preferably, when P₁ and P₂ are polyethylene glycol with a single molecular weight, the molecular weight is 88-4400 Da, and more preferably, the molecular weight of P₁ and P₂ is 176-1056 Da;
preferably, when P₁ and P₂ are polyethylene glycol with a non-single molecular weight, the molecular weight is 1000 Da-40 kDa;
more preferably, the molecular weight of P₁ and P₂ is 2000 Da-10 kDa.

Preferably, L₁ is a linking group selected from one or a combination of two or more of linear or branched C₁₋₁₂ alkylene, C₆₋₁₂ arylene, C₃₋₁₂ cycloalkylene, —S—,

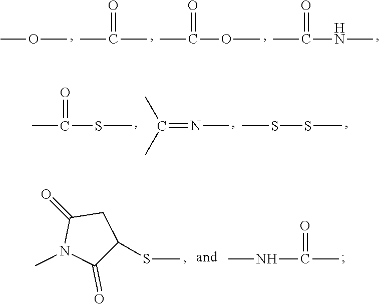

an optional H atom on the linear or branched C₁₋₁₂ chain alkylene, C₆₋₁₂ arylene, or C₃₋₁₂ cycloalkylene is substituted with one or a combination of two or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl, substituted or unsubstituted heterocyclylalkyl,

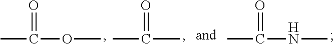

L₂ is a linking group selected from one or a combination of two or more of linear or branched C₁₋₁₂ alkylene, C₆₋₁₂ arylene, C₃₋₁₂ cycloalkylene, —S—,

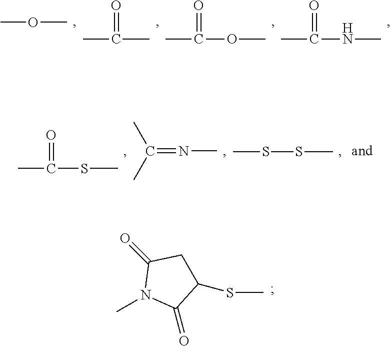

an optional H atom on the linear or branched C₁₋₁₂ chain alkylene, C₆₋₁₂ arylene, or C₃₋₁₂ cycloalkylene is substituted with one or a combination of two or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl, substituted or unsubstituted heterocyclylalkyl,

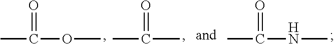

preferably, L₁ is an amide bond, a hydrazone bond, or a thiol-maleimide bond;

more preferably, L₁ is an amide bond;

preferably, L₂ is a disulfide bond or a thiol-maleimide bond;

more preferably, L₂ is a disulfide bond.

Preferably, A₁ is a linking group connecting P₁ and P₂, and is selected from one or a combination of two or more of linear or branched C₁₋₁₂ alkylene, C₆₋₁₂ arylene, C₃₋₁₂ cycloalkylene, —S—,

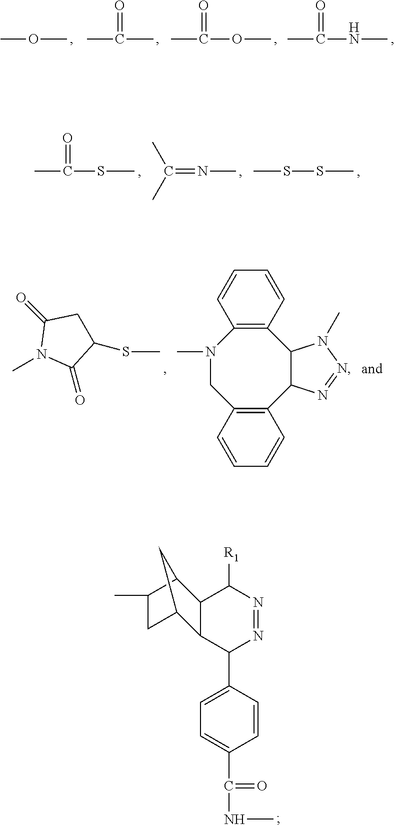

an optional H atom on the linear or branched C₁₋₁₂ chain alkylene, C₆₋₁₂ arylene, or C₃₋₁₂ cycloalkylene is substituted with one or a combination of two or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl, substituted or unsubstituted heterocyclylalkyl,

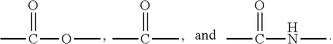

Preferably, the antibody-nucleic acid drug conjugate has a general formula (IV):

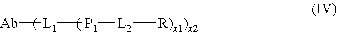

wherein L₁ is a linking group selected from one or a combination of two or more of linear or branched C₁₋₁₂ alkylene, C₆₋₁₂ arylene, C₃₋₁₂ cycloalkylene, —S—,

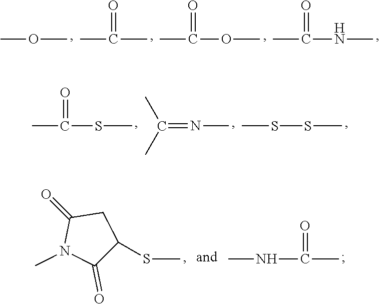

preferably, P₁ are identical or different polyethylene glycol residues;

preferably, when P₁ is a polyethylene glycol with a single molecular weight, the molecular weight is 88-4400 Da, and more preferably, the molecular weight of P₁ is 176-1056 Da;

preferably, when P₁ is a polyethylene glycol with a non-single molecular weight, the molecular weight is 1000 Da-40 kDa; more preferably, the molecular weight of P₁ is 2000 Da-10 kDa;

L₂ is a linking group selected from one or a combination of two or more of linear or branched C₁₋₁₂ alkylene, C₆₋₁₂ arylene, C₃₋₁₂ cycloalkylene, —S—,

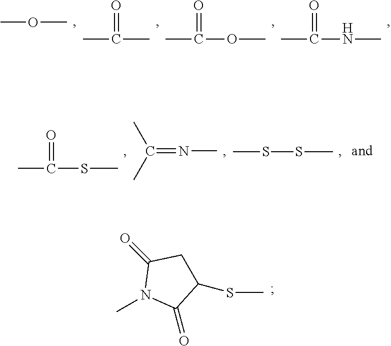

x1 is an integer of 1-144; preferably, x1 is an integer of 1-9; more preferably, x1 is an integer of 1-3;

x2 is an integer of 1-8; preferably, x2 is an integer of 1-2.

Preferably, the antibody-nucleic acid drug conjugate has the following structure:

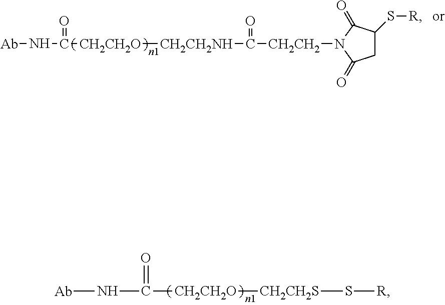

wherein n1 is selected from integers of 4-100, preferably 4-24; n1 may be a constant value or an average value.

Preferably, Ab is selected from a monoclonal antibody, a polyclonal antibody, an antibody fragment, and an antibody fusion fragment.

The antibody may be a single domain antibody or a single chain antibody.

Further preferably, Ab is a monoclonal antibody; more preferably, the monoclonal antibody is reactive to an antigen or an epitope thereof associated with a cancer, a malignant cell, an infectious organism, or an autoimmune disease.

In one specific embodiment of the present invention, Ab is selected from an anti-HER2 antibody, an anti-EGFR antibody, an anti-PMSA antibody, an anti-VEGFR antibody, an anti-CD30 antibody, an anti-CD22 antibody, an anti-CD56 antibody, an anti-CD29 antibody, an anti-GPNMB antibody, an anti-CD138 antibody, an anti-CD74 antibody, an anti-ENPP3 antibody, an anti-Nectin-4 antibody, an anti-EGFRVIII antibody, an anti-SLC44A4 antibody, an anti-mesothelin antibody, an anti-ET8R antibody, an anti-CD37 antibody, an anti-CEACAM5 antibody, an anti-CD70 antibody, an anti-MUC16 antibody, an anti-CD79b antibody, an anti-MUC16 antibody, an anti-Muc1 antibody, an anti-CD3 antibody, an anti-CD28 antibody, an anti-CD38 antibody, an anti-CD19 antibody, an anti-PD-L1 antibody, an anti-4-1BB antibody, etc. In one specific embodiment of the present invention, the antibody-nucleic acid drug conjugate has the following structure:

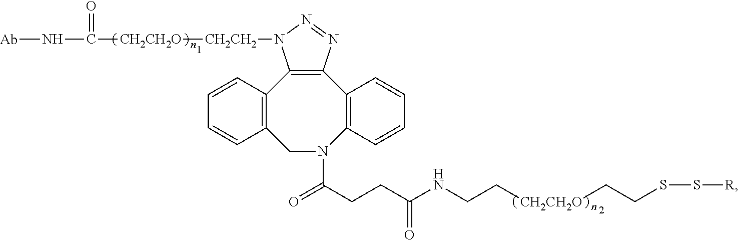

wherein n₁ and n₂ are independently selected from integers of 4-100, preferably 4-24; n₁ and n₂ may be constant values or average values.

In an eighth aspect of the present invention, provided is use of the interfering RNA described above, the delivery system described above, the cell described above, the medicament or the kit described above, the lipid nanoparticle described above, or the antibody-nucleic acid drug conjugate described above, comprising:
A) use in preparing a medicament for treating and/or preventing a TOP1 expression-associated disease;
B) use in inhibiting TOP1 expression;
C) use in treating and/or preventing a TOP1 expression-associated disease.

Preferably, the interfering RNA, the delivery system described above, the cell described above, the medicament or the kit described above, the lipid nanoparticle described above, or the antibody-nucleic acid drug conjugate described above used in the treatment has a working concentration of 0.01-1000 nM, e.g., 0.01, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, or 1000 nM.

The TOP1 expression-associated disease is a tumor with TOP1 expression or high TOP1 expression.

The TOP1 expression-associated disease is a tumor marked by TOP1 expression.

The TOP1 expression-associated disease is a tumor that needs to be treated by inhibiting TOP1 expression.

Preferably, the TOP1 expression-associated disease comprises a tumor. Further preferably, the tumor comprises a digestive system tumor (oral cancer, tongue cancer, esophageal cancer, gastric cancer, liver cancer, pancreatic cancer, colorectal cancer, etc.), a nervous system tumor (glioma (e.g., neuroglioma), neuroepithelioma, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urinary system tumor (e.g., prostate cancer, renal cell carcinoma, bladder cancer, etc.), a reproductive system tumor (breast cancer, cervical cancer, ovarian cancer, placental choriocarcinoma, etc.), a hematological and lymphatic system tumor (multiple myeloma, mesothelioma, myeloma, myelodysplastic syndrome, lymphoma (e.g., non-Hodgkin's lymphoma, Hodgkin's lymphoma, and cutaneous T-cell lymphoma), leukemia (e.g., chronic myeloid leukemia, acute myelogenous leukemia, chronic lymphocytic leukemia, etc.), thymus cancer, etc.), a cutaneous system tumor (e.g., skin cancer, epidermoid cancer, melanoma, etc.), etc., as well as rhabdomyosarcoma, head and neck cancer, etc.

Further preferably, the tumor is selected from a digestive system tumor, a cutaneous system tumor, and a urinary system tumor.

In one specific embodiment of the present invention, the tumor is selected from colon cancer, liver cancer, prostate cancer, and melanoma.

In a ninth aspect of the present invention, provided is a method for inhibiting TOP1 expression, comprising adding the interfering RNA described above, the delivery system described above, the cell described above, the lipid nanoparticle described above, the medicament or the kit described above, or the antibody-nucleic acid drug conjugate described above.

Preferably, the method inhibits TOP1 expression in a cell, and the cell is a tumor cell.

Preferably, the method comprises delivering the interfering RNA described above to a cell. The delivery is performed using the delivery system described above.

In a tenth aspect of the present invention, provided is a method for treating a TOP1 expression-associated disease, comprising administering to a subject a therapeutically effective amount of the interfering RNA described above, the delivery system described above, the lipid nanoparticle described above, the cell described above, the medicament or the kit described above, or the antibody-nucleic acid drug conjugate described above.

Preferably, the TOP1 expression-associated disease comprises a tumor. Further preferably, the tumor comprises a digestive system tumor (oral cancer, tongue cancer, esophageal cancer, gastric cancer, liver cancer, pancreatic cancer, colorectal cancer, etc.), a nervous system tumor (glioma (e.g., neuroglioma), neuroepithelioma, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urinary system tumor (e.g., prostate cancer, renal cell carcinoma, bladder cancer, etc.), a reproductive system tumor (breast cancer, cervical cancer, ovarian cancer, placental choriocarcinoma, etc.), a hematological and lymphatic system tumor (multiple myeloma, mesothelioma, myeloma, myelodysplastic syndrome, lymphoma (e.g., non-Hodgkin's lymphoma, Hodgkin's lymphoma, and cutaneous T-cell lymphoma), leukemia (e.g., chronic myeloid leukemia, acute myelogenous leukemia, chronic lymphocytic leukemia, etc.), thymus cancer, etc.), a cutaneous system tumor (e.g., skin cancer, epidermoid cancer, melanoma, etc.), etc., as well as rhabdomyosarcoma, head and neck cancer, etc.

In an eleventh aspect of the present invention, provided is a method for treating a tumor, comprising administering to a subject a therapeutically effective amount of the interfering RNA described above, the delivery system described above, the cell described above, the lipid nanoparticle described above, the medicament or the kit described above, or the antibody-nucleic acid drug conjugate described above.

The TOP1 expression-associated disease is a tumor with TOP1 expression or high TOP1 expression.

The TOP1 expression-associated disease is a tumor marked by TOP1 expression.

The TOP1 expression-associated disease is a tumor that needs to be treated by inhibiting TOP1 expression.

Preferably, the tumor comprises a digestive system tumor (oral cancer, tongue cancer, esophageal cancer, gastric cancer, liver cancer, pancreatic cancer, colorectal cancer, etc.), a nervous system tumor (glioma (e.g., neuroglioma), neuroepithelioma, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urinary system tumor (e.g., prostate cancer, renal cell carcinoma, bladder cancer, etc.), a reproductive system tumor (breast cancer, cervical cancer, ovarian cancer, placental choriocarcinoma, etc.), a hematological and lymphatic system tumor (multiple myeloma, mesothelioma, myeloma, myelodysplastic syndrome, lymphoma (e.g., non-Hodgkin's lymphoma, Hodgkin's lymphoma, and cutaneous T-cell lymphoma), leukemia (e.g., chronic myeloid leukemia, acute myelogenous leukemia, chronic lymphocytic leukemia, etc.), thymus cancer, etc.), a cutaneous system tumor (e.g., skin cancer, epidermoid cancer, melanoma, etc.), etc., as well as rhabdomyosarcoma, head and neck cancer, etc.

Further preferably, the tumor is selected from a digestive system tumor, a cutaneous system tumor, and a urinary system tumor.

In one specific embodiment of the present invention, the tumor is selected from colon cancer, liver cancer, prostate cancer, and melanoma.

The “interfering RNA” as described herein includes a single-stranded RNA (ssRNA, e.g., mature miRNA) or a double-stranded RNA (dsRNA, e.g., siRNA, shRNA, aiRNA, or pre-miRNA), which is capable of reducing or inhibiting the expression of a target gene or sequence (e.g., by mediating degradation and/or inhibition of an mRNA complementary to the interfering RNA in sequence to affect the mRNA translation) when the interfering RNA and the target gene or sequence are in the same cell.

The siRNA is a small interfering RNA, and each strand of the siRNA molecule comprises nucleotides of about 15 nt to about 60 nt in length (e.g., nucleotides of about 15-60 nt, 15-50 nt, 15-40 nt, 15-30 nt, 15-25 nt, or 19-25 nt in length, or nucleotides of 15, 16, 17, 18, 19, 20, 21, 22, 23, 24, or 25 nt in length). In one specific embodiment, the siRNA may be chemically synthesized. The siRNA molecule of the present invention can silence the expression of a target sequence in vitro and/or in vivo. In some embodiments, the siRNA may not contain modified nucleotides; in other embodiments, the siRNA comprises at least one modified nucleotide. For example, the siRNA comprises one, two, three, four, five, six, seven, eight, nine, ten, or more modified nucleotides in the double-stranded region. The dsRNA or pre-RNA molecules include any precursor molecule that is processed in vivo by an endonuclease to produce an active siRNA. The shRNA is a small hairpin RNA or a short hairpin RNA comprising short RNA sequences producing tight hairpin turns that can be used to silence gene expression via RNA interference. The shRNA hairpin structure can be processed in a cell to form an siRNA. The miRNA (microRNA) is a single-stranded RNA molecule regulating gene expression with about 21-23 nucleotides in length.

“Inhibiting the expression of a target gene” as described herein refers to the ability of the interfering RNA (e.g., siRNA) of the present invention to silence, reduce, or inhibit the expression of a target gene (e.g., TOP1 gene).

The term “comprise”, “comprising”, or “include” as described herein is an open-ended description that includes the specified ingredients or steps as described, as well as other specified ingredients or steps that do not substantially affect the technical effect. When the term is used to describe a sequence of a protein or nucleic acid, the protein or nucleic acid may consist of the sequence, or may have additional amino acids or nucleotides at one or two ends of the protein or nucleic acid, while retaining the same or similar activity as the original sequence.

The term “tumor” as described herein may be any adverse cell proliferation (or any disease that manifests itself as adverse cell proliferation) or neoplasm, or increased predisposition or risk for adverse cell proliferation, neoplasm, or a tumor. The tumor may be benign or malignant, and may also be primary or secondary (metastatic). The neoplasm may be any abnormal growth or proliferation of cells and may be located in any tissue. Examples of the tissue include adrenal gland, adrenal medulla, anus, appendix, bladder, blood, bone, bone marrow, brain, mammary gland, cecum, central nervous system (including or excluding brain), cerebellum, cervix, colon, duodenum, endometrium, epithelial cells (e.g., renal epithelial cells), gallbladder, esophagus, glial cells, heart, ileum, jejunum, kidney, lacrimal gland, larynx, liver, lung, lymph, lymph nodes, lymphoblasts, maxilla, mediastinum, mesenterium, myometrium, nasopharynx, omentum, oral cavity, ovary, pancreas, parotid gland, peripheral nervous system, peritoneum, pleura, prostate, salivary glands, sigmoid colon, skin, small intestine, soft tissue, spleen, stomach, testis, thymus, thyroid, tongue, tonsils, trachea, uterus, vulva, and white blood cells. Further preferably, the tumor comprises a digestive system tumor (oral cancer, tongue cancer, esophageal cancer, gastric cancer, liver cancer, pancreatic cancer, colorectal cancer, etc.), a nervous system tumor (glioma (e.g., neuroglioma), neuroepithelioma, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urinary system tumor (e.g., prostate cancer, renal cell carcinoma, bladder cancer, etc.), a reproductive system tumor (breast cancer, cervical cancer, ovarian cancer, placental choriocarcinoma, etc.), a hematological and lymphatic system tumor (multiple myeloma, mesothelioma, myeloma, myelodysplastic syndrome, lymphoma (e.g., non-Hodgkin's lymphoma, Hodgkin's lymphoma, and cutaneous T-cell lymphoma), leukemia (e.g., chronic myeloid leukemia, acute myelogenous leukemia, chronic lymphocytic leukemia, etc.), thymus cancer, etc.), a cutaneous system tumor (e.g., skin cancer, epidermoid cancer, melanoma, etc.), etc., as well as rhabdomyosarcoma, head and neck cancer, etc.

The term “subject” as described herein may be a human or non-human mammal, and the non-human mammal may be a wild animal, a zoo animal, an economic animal, a companion animal, a laboratory animal, etc. Preferably, the non-human mammal includes, but is not limited to, a pig, a cow, a sheep, a horse, a donkey, a fox, a raccoon dog, a mink, a camel, a dog, a cat, a rabbit, a rodent (e.g., a rat, a mouse, a guinea pig, a hamster, a gerbil, a chinchilla, and a squirrel), a monkey, etc.

The term “treating” as described herein refers to slowing, interrupting, arresting, controlling, stopping, reducing, or reversing the progression or severity of a sign, symptom, disorder, condition, or disease after the disease has begun to progress, but it does not necessarily involve the complete elimination of all disease-related signs, symptoms, conditions, or disorders.

The term “preventing” as described herein refers to the practice of preventing or delaying the onset of a disease or condition or symptom in the body.

The term “effective amount” as described herein refers to an amount or dose of the product of the present invention that provides a desired effect (e.g., treating or preventing a TOP1 expression-associated disease or inhibiting TOP1 expression) after administration to a subject or a cell or organ thereof in a single dose or multiple doses.

The term “lipid” as described herein refers to a group of organic compounds that include, but are not limited to, esters of fatty acids and are generally characterized by being poorly soluble in water, but soluble in many organic solvents.

The term “cationic lipid” as described herein refers to a lipid molecule capable of being positively charged.

The term “neutral lipid” as described herein refers to an uncharged non-phosphoglyceride lipid molecule.

The term “polyethylene glycol lipid” as described herein refers to a molecule comprising a lipid portion and a polyethylene glycol portion.

The term “lipid nanoparticle” as described herein refers to a particle with at least one dimension in the nanometer scale, which comprises at least one lipid.

The term “delivery system” as described herein refers to a formulation or composition that regulates the spatial, temporal, and dose distribution of a bioactive ingredient in an organism.

### BRIEF DESCRIPTION OF THE DRAWINGS

Hereinafter, examples of the present invention will be described in detail with reference to the accompanying drawings, in which:

FIG. 1 shows the mass spectra of the sense strand (ss) and the antisense strand (as) of siTOP1-267.

FIG. 2 shows the mass spectra of the sense strand (ss) and the antisense strand (as) of siTOP1-628.

FIG. 3 shows the mass spectra of the sense strand (ss) and the antisense strand (as) of siTOP1-896.

FIG. 4 shows the mass spectra of the sense strand (ss) and the antisense strand (as) of siTOP1-1261.

FIG. 5 shows the mass spectra of the sense strand (ss) and the antisense strand (as) of siTOP1-1282.

FIG. 6 shows the mass spectra of the sense strand (ss) and the antisense strand (as) of siTOP1-1357.

FIG. 7 shows the mass spectra of the sense strand (ss) and the antisense strand (as) of siTOP1-1459.

FIG. 8 shows the mass spectra of the sense strand (ss) and the antisense strand (as) of siTOP1-1530.

FIG. 9 shows the mass spectra of the sense strand (ss) and the antisense strand (as) of siTOP1-1604.

FIG. 10 shows the mass spectra of the sense strand (ss) and the antisense strand (as) of siTOP1-1630.

FIG. 11 shows the mass spectra of the sense strand (ss) and the antisense strand (as) of siTOP1-1679.

FIG. 12 shows the mass spectra of the sense strand (ss) and the antisense strand (as) of siTOP1-1776.

FIG. 13 shows the mass spectra of the sense strand (ss) and the antisense strand (as) of siTOP1-2273.

FIG. 14 shows the mass spectra of the sense strand (ss) and the antisense strand (as) of siTOP1-2585.

FIG. 15 shows the mass spectra of the sense strand (ss) and the antisense strand (as) of siTOP1-2898.

FIG. 16 shows the detection of the inhibitory effect of siTOP1 on TOP1 mRNA in A375 cells.

FIG. 17 shows the inhibitory effects of siTOP1 on A375 cell proliferation.

FIG. 18A shows the dose-dependence of siTOP1-1604 in human prostate cancer cells.

FIG. 18B shows the dose-dependence of siTOP1-1604 in human colon cancer cells.

FIG. 19 shows the multimodal particle size distribution profile of LNP-siTOP1-1604.

FIG. 20 shows the detection of the inhibitory effect of LNP-siTOP1-1604 on TOP1 mRNA in various tumor cells.

FIG. 21 shows the inhibitory effect of a single transfection of LNP-siTOP1-1604 on PC-3 cell proliferation.

FIG. 22A shows the in vivo inhibitory effect of LNP-siTOP1-1604 on tumor volumes in mice.

FIG. 22B shows the tumor growth inhibition on different days after the LNP-siTOP1-1604 treatment.

FIG. 23A shows the inhibitory effect of LNP-siTOP1-1604 on the colony formation of HCT116.

FIG. 23B shows the inhibitory effect of LNP-siTOP1-1604 on the areas and numbers of HCT116 colonies.

FIG. 24 shows the inhibitory effects of the modified sequences of siTOP1 on TOP1 mRNA.

### DETAILED DESCRIPTION

The technical solutions in the examples of the present invention will be described clearly and completely below with reference to the accompanying drawings in the examples of the present invention. It is obvious that the described examples are only a part of the examples of the present invention, rather than all of them. Based on the examples of the present invention, all other examples obtained by those of ordinary skill in the art without creative work shall fall within the protection scope of the present invention.

### Example 1: siRNA Design and Synthesis

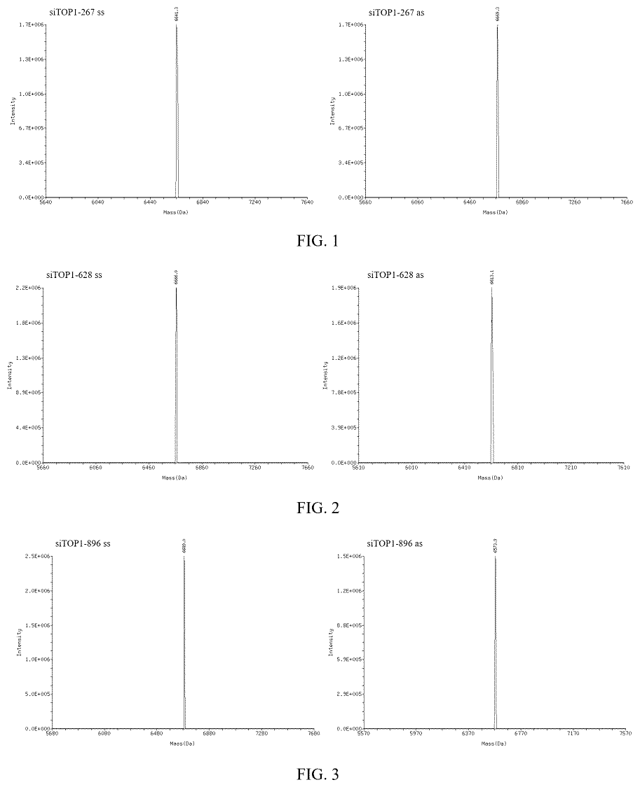

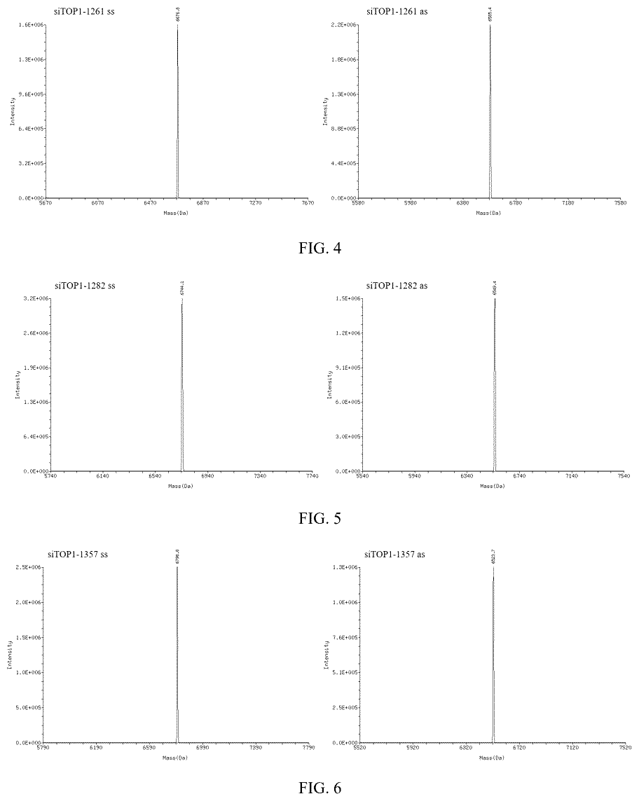

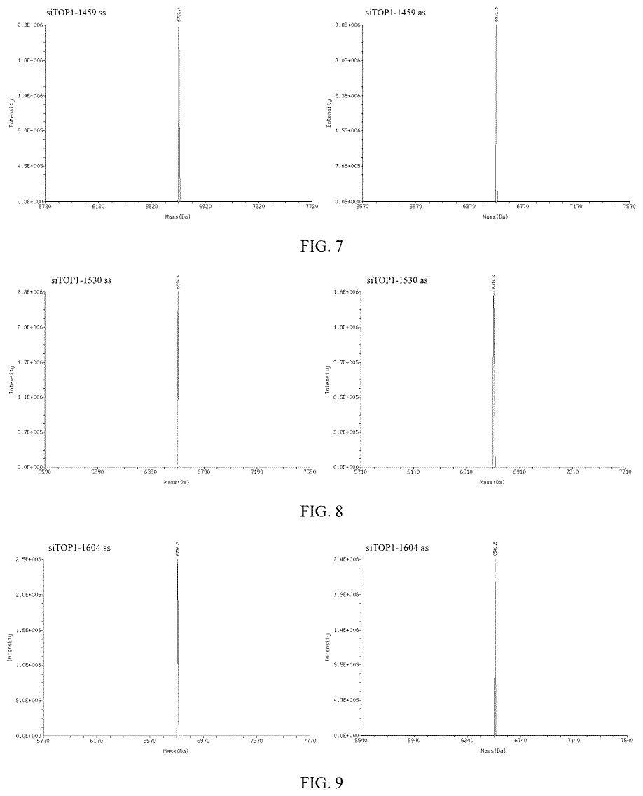

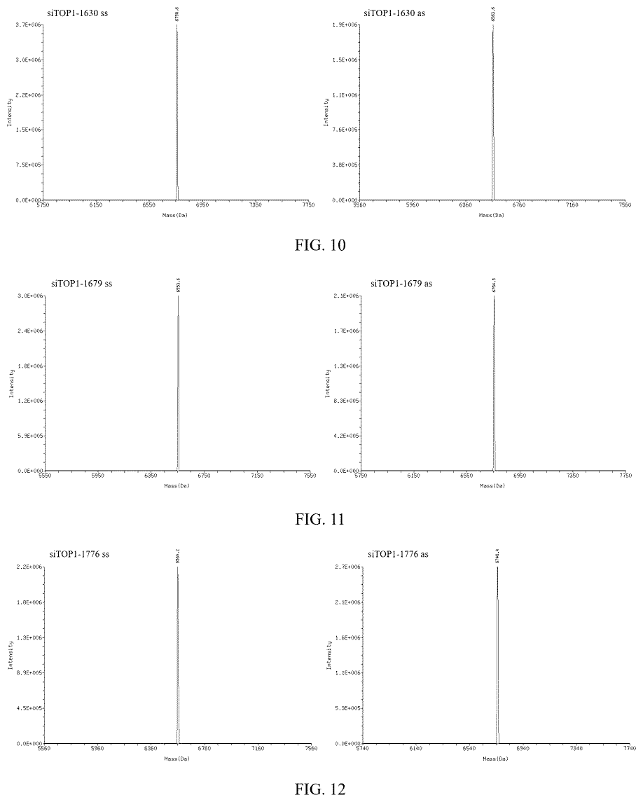

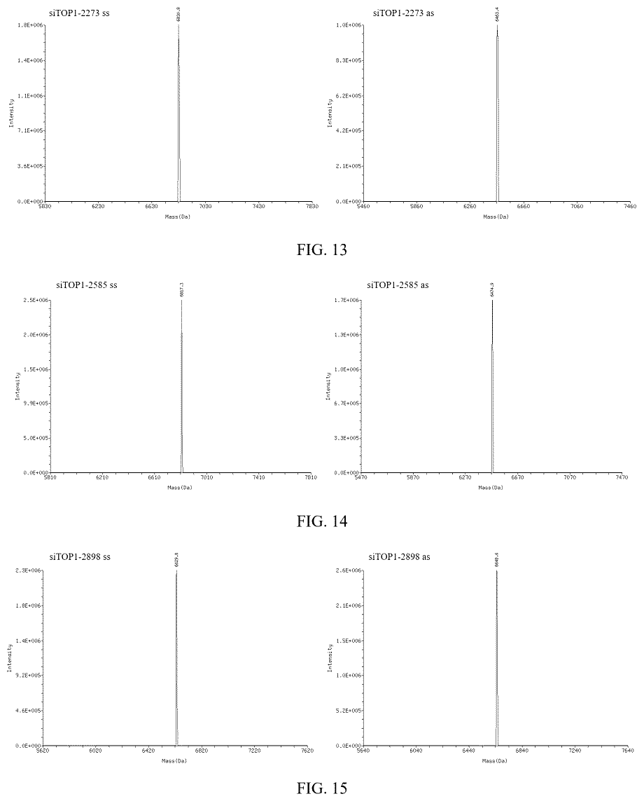

The siRNA targeting topoisomerase I (TOP) was designed and synthesized to reduce TOP expression level and treat TOP1 expression-associated diseases. After screening, sequences shown in Table 1 were obtained, and the mass spectrometry results are shown in FIGS. 1-15.

TABLE 1
TOP1 target sequences and siRNA sequences

| Name | Target sequence | Sense strand | Antisense strand |
|---|---|---|---|
| siTOP1- | CAACGATTCCCAGATCG | CAACGAUUCCCAGAUCGAA | UUCGAUCUGGGAAUCGUU |
| 267 | AA (SEQ ID NO: 1) | dTdT (SEQ ID NO: 16) | GdTdT (SEQ ID NO: 31) |
| siTOP1- | CCTCCTAAAGAGGATATA | CCUCCUAAAGAGGAUAUA | UUAUAUCCUCUUUAGGAG |
| 628 | A (SEQ ID NO: 2) | AdTdT (SEQ ID NO: 17) | GdTdT (SEQ ID NO: 32) |
| siTOP1- | GGAAATTCCTAGAACAT | GGAAAUUCCUAGAACAUA | UUAUGUUCUAGGAAUUUC |
| 896 | AA (SEQ ID NO: 3) | AdTdT (SEQ ID NO: 18) | CdTdT (SEQ ID NO: 33) |
| siTOP1- | GGATTCTGTATTATGGAT | GGAUUCUGUAUUAUGGAU | UAUCCAUAAUACAGAAUC |
| 1261 | A (SEQ ID NO: 4) | AdTdT (SEQ ID NO: 19) | CdTdT (SEQ ID NO: 34) |
| siTOP1- | CACAAAGAGAGGATTGC | CACAAAGAGAGGAUUGCU | UAGCAAUCCUCUCUUUGU |
| 1282 | TA (SEQ ID NO: 5) | AdTdT (SEQ ID NO: 20) | GdTdT (SEQ ID NO: 35) |
| siTOP1- | GGCATGCTGAAGAGACG | GGCAUGCUGAAGAGACGA | UUCGUCUCUUCAGCAUGCC |
| 1357 | AA (SEQ ID NO: 6) | AdTdT (SEQ ID NO: 21) | dTdT (SEQ ID NO: 36) |
| siTOP1- | CGGCATGATAACAAGGT | CGGCAUGAUAACAAGGUU | UAACCUUGUUAUCAUGCC |
| 1459 | TA (SEQ ID NO: 7) | AdTdT (SEQ ID NO: 22) | GdTdT (SEQ ID NO: 37) |
| siTOP1- | GCTTAACCCTAGTTCAC | GCUUAACCCUAGUUCACGA | UCGUGAACUAGGGUUAAG |
| 1530 | GA (SEQ ID NO: 8) | dTdT (SEQ ID NO: 23) | CdTdT (SEQ ID NO: 38) |
| siTOP1- | GTGTGGACAAGATCCGG | GUGUGGACAAGAUCCGGA | UUCCGGAUCUUGUCCACAC |
| 1604 | AA (SEQ ID NO: 9) | AdTdT (SEQ ID NO: 24) | dTdT (SEQ ID NO: 39) |
| siTOP1- | CGAGAAGACTGGAAGTC | CGAGAAGACUGGAAGUCC | UGGACUUCCAGUCUUCUC |
| 1630 | CA (SEQ ID NO: 10) | AdTdT (SEQ ID NO: 25) | GdTdT (SEQ ID NO: 40) |
| siTOP1- | CCCTGTACTTCATCGACA | CCCUGUACUUCAUCGACAA | UUGUCGAUGAAGUACAGG |
| 1679 | A (SEQ ID NO: 11) | dTdT (SEQ ID NO: 26) | GdTdT (SEQ ID NO: 41) |
| siTOP1- | GCACATCAATCTACACC | GCACAUCAAUCUACACCCA | UGGGUGUAGAUUGAUGUG |
| 1776 | CA (SEQ ID NO: 12) | dTdT (SEQ ID NO: 27) | CdTdT (SEQ ID NO: 42) |
| siTOP1- | AGGATGCAAAGACGAA | AGGAUGCAAAGACGAAGA | UUCUUCGUCUUUGCAUCC |
| 2273 | GAA (SEQ ID NO: 13) | AdTdT (SEQ ID NO: 28) | UdTdT (SEQ ID NO: 43) |
| siTOP1- | GGTTTGGGAAAGATGGA | GGUUUGGGAAAGAUGGAU | UAUCCAUCUUUCCCAAACC |
| 2585 | TA (SEQ ID NO: 14) | AdTdT (SEQ ID NO: 29) | dTdT (SEQ ID NO: 44) |
| siTOP1- | CGATTATATTGGTGCGTT | CGAUUAUAUUGGUGCGUU | AAACGCACCAAUAUAAUC |
| 2898 | T (SEQ ID NO: 15) | UdTdT (SEQ ID NO: 30) | GdTdT (SEQ ID NO: 45) |

### Example 2: Inhibitory Effects of siTOP1 on TOP1 mRNA in Human Melanoma Cells

### 1. Cell Transfection

1) On the day before transfection, A375 melanoma cells were seeded into a 24-well plate at 6×10⁴ to 8×10⁴ cells/well, the culture medium being 500 μL of DMEM containing 10% FBS and 1% Penicillin-Streptomycin, and the plate was placed in an incubator with 5% CO₂ at 37° C. for 24 h to allow the cells to adhere to the wall. Three replicate wells were made for each group.
2) On the day of transfection, the culture medium was changed to 450 μL of DMEM culture medium without antibiotics (containing 10% FBS).
3) The siRNAs were diluted with 25 μL of Opti-MEM culture medium; the siNC (the siNC (reference: O Sordet et al., 2008) having the target sequence of 5′-TTCTCCGAACGTGTCACGT-3′ (SEQ ID NO: 46)) at the same concentration was the negative control group; Lipofectamine™ RNAiMAX (Lipo) transfection reagent was diluted with another 25 μL of Opti-MEM culture medium and left to stand at room temperature for 5 min; and the two dilutions were uniformly mixed and left to stand at room temperature for 5 min.
4) 50 μL of the mixed solution described above was added to each well to obtain a final transfection volume of 500 μL. and the working concentration of the siRNA was 50 nM.
5) After the transfection, the cells were cultured for 48 h and then collected.

### 2. RNA Extraction

1) RNA was extracted using an Eastep™ Super total RNA extraction kit; 300 μL of RNA lysis solution and 300 μL of RNA diluent were added; and the mixture was uniformly mixed and then transferred to a 1.5 mL centrifuge tube.
2) The mixture was centrifuged at 14000 g for 5 min, and the supernatant was carefully pipetted into a new 1.5 mL centrifuge tube.
3) A 0.5-fold volume of absolute ethanol was added, and the mixture was uniformly mixed by allowing the tube to be repeatedly upside down 15-20 times. Subsequently, the mixture was transferred to a centrifuge column and centrifuged at 12000 g for 1 min. The filtrate was discarded.
4) 600 μL of RNA wash buffer was added, and the mixture was centrifuged at 12000 g for 1 min. The filtrate was discarded.
5) 50 μL of DNase I incubation buffer was added, followed by DNA digestion at room temperature for 15 min.
6) 600 μL of RNA wash buffer was added, the mixture was centrifuged at 12000 g for 1 min, and the filtrate was discarded. This procedure was repeated once.
7) The mixture was centrifuged at 14000 g for 2 min to remove the residual RNA wash buffer, and then the centrifuge column was placed in a collection tube.
8) An appropriate amount of RNase-free H₂O was added, and the mixture was left to stand at room temperature for 12 min and centrifuged at 14000 g for 1 min. The collected RNA was preserved at −70° C. for later use.

### 3. Detection of Gene Expression Level

1) The mass and concentration of the RNA were detected using NanoDrop™, and an RNA template was prepared as follows: RNase-free H₂O was added to 1 μg of RNA to a volume of 10 μL; the mixture was placed at 70° C. for 5 min and then placed in an ice bath for 5 min for later use.
2) A reaction system was established according to Table 2:

TABLE 2
Reaction system

| Reagent | Amount of use |
|---|---|
| GoScript ™ReactionBuffer, Oliga (dT) | 4 μL |
| GoScript ™ Enzyme Mix | 2 μL |
| RNA template | 10 μL |
| RNase-free H₂O | Make up to 20 μL |
| Total | 20 μL |

3) The reaction system described above was uniformly mixed and centrifuged to collect the liquid at the bottom of a tube, and the mixture was subjected to the procedures of 25° C. for 5 min, 42° C. for 60 min, and 72° C. for 15 min, thereby giving the cDNA product.
4) A Q-PCR system was prepared according to Table 3, and the level of TOP1 gene was detected:

TABLE 3
Reaction system

| Reagent | Amount of addition |
|---|---|
| 2 × TB Green ® Premix Ex Taq ™ II | 10 μL |
| Forward primer (10 μM) | 0.5 μL |
| Reverse primer (10 μM) | 0.5 μL |
| cDNA (50-100 ng) | 1.2 μL |
| RNase-free H₂O | 7.8 μL |
| Total | 20 μL |

GAPDH was used as an endogenous reference, and the sequences of the primers used for detection are as follows:

GAPDH-F:
(SEQ ID NO: 47)
TCTGACTTCAACAGCGACAC;
GAPDH-R:
(SEQ ID NO: 48)
GCCAAATTCGTTGTCATACC;
TOP1-F:
(SEQ ID NO: 49)
CTGTAGCCCTGTACTTCATCG;
TOP1-R:
(SEQ ID NO: 50)
CTACCACATATTCCTGACCATCC.

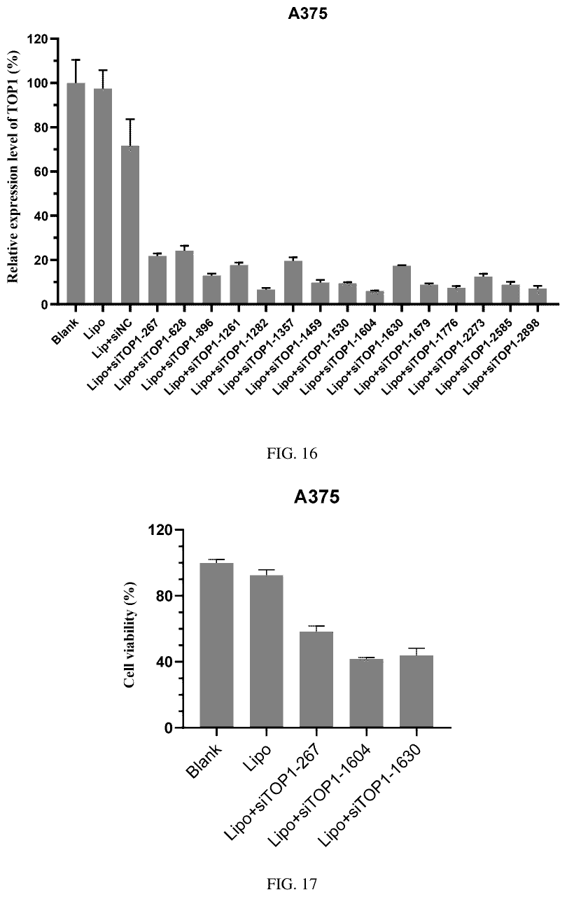

The results demonstrate that in the A375 cells cultured for 48 h after siRNA transfection, the TOP1-targeting siRNAs designed in Example 1 were all capable of inhibiting TOP1 expression when compared to Mock and siNC. Among them, siTOP1-1282 (inhibition by 93.4%), siTOP1-1459 (inhibition by 90.1%), siTOP1-1530 (inhibition by 90.6%), siTOP1-1604 (inhibition by 94.0%), siTOP1-1679 (inhibition by 91.1%), siTOP1-1776 (inhibition by 92.6%), siTOP1-2585 (inhibition by 91.2%), and siTOP1-2898 (inhibition by 93.0%) had better inhibitory effects (Table 4 and FIG. 16).

TABLE 4
Detection of the inhibitory effect of siTOP1 on the gene in the cells

Relative expression level of gene

| | | Standard |
|---|---|---|
| | Mean | deviation |
| A375 | value | (SD) |
| Blank | 100.00 | 10.47 |
| Lipo | 97.50 | 8.28 |
| Lipo + siNC | 71.61 | 12.00 |
| Lipo + siTOP1-267 | 21.85 | 1.15 |
| Lipo + siTOP1-628 | 24.27 | 2.21 |
| Lipo + siTOP1-896 | 13.05 | 0.78 |
| Lipo + siTOP1-1261 | 17.75 | 1.14 |
| Lipo + siTOP1-1282 | 6.64 | 0.76 |
| Lipo + siTOP1-1357 | 19.62 | 1.63 |
| Lipo + siTOP1-1459 | 9.89 | 1.13 |
| Lipo + siTOP1-1530 | 9.44 | 0.58 |
| Lipo + siTOP1-1604 | 5.97 | 0.21 |
| Lipo + siTOP1-1630 | 17.34 | 0.35 |
| Lipo + siTOP1-1679 | 8.86 | 0.55 |
| Lipo + siTOP1-1776 | 7.41 | 0.85 |
| Lipo + siTOP1-2273 | 12.54 | 1.27 |
| Lipo + siTOP1-2585 | 8.84 | 1.37 |
| Lipo + siTOP1-2898 | 7.05 | 1.26 |

### Example 3: Inhibitory Effects of siTOP1 on Human Melanoma Cell Proliferation

### 1. Cell Transfection

1) On the day before transfection, A375 melanoma cells were seeded into a 96-well plate at 2500-3000 cells/well, the culture medium being a total volume of 200 μL of DMEM culture medium containing 7.5% FBS and 1% Penicillin-Streptomycin, and the plate was placed in an incubator with 5% CO₂ at 37° C. for 24 h to allow the cells to adhere to the wall. 5 replicate wells were made for each group.
2) On the day of transfection, the culture medium was changed to 90 μL of reduced serum culture medium without antibiotics and containing 7.5% FBS.
3) The siRNAs were diluted with an appropriate amount of Opti-MEM culture medium; Lipofectamine™ RNAiMAX (Lipo) transfection reagent was diluted with another appropriate amount of Opti-MEM culture medium and left to stand at room temperature for 5 min; and the two dilutions were uniformly mixed and left to stand at room temperature for 5 min.
4) 10 μL of the mixed solution described above was added to each well to obtain a final transfection volume of 100 μL, and the working concentration of the siRNA was 50 nM.
5) The cells were cultured in the incubator with 5% CO₂ at 37° C. for 72 h.

### 2. Cell Proliferation Inhibition Assay

After 72 h of transfection, 10 μL of CCK-8 solution was added to each well, and the cells were cultured in the incubator with 5% CO₂ at 37° C. for another 4 h. The absorbance value at 450 nm was measured using a microplate reader. Cell viability was calculated according to the following formula:

$$Cell viability ( \% ) = 100 \times OD 45 {0}_{e x p e r i m ental group} / OD {450}_{B l a n k}$$

The results demonstrate that the siRNAs designed in Example 1 were capable of inhibiting A375 cell proliferation. Exemplary results are shown in Table 5 and FIG. 17, where siTOP1-267 had inhibition by about 42%, siTOP1-1604 had inhibition by about 58%, and siTOP1-1630 had inhibition by about 56% compared to Blank.

TABLE 5
Inhibitory effects of siTOP1 on A375 cell proliferation

Cell viability (%)

| | | Standard |
|---|---|---|
| | Mean | deviation |
| A375 | value | (SD) |
| Blank | 100.00 | 2.09 |
| Lipo | 92.56 | 3.17 |
| Lipo + siTOP1-267 | 58.27 | 3.51 |
| Lipo + siTOP1-1604 | 41.65 | 1.04 |
| Lipo + siTOP1-1630 | 43.84 | 4.39 |

### Example 4: Inhibitory Effects of siTOP1 on TOP1 mRNA in Human Prostate Cancer Cells and Human Colon Cancer Cells

A transfection system was prepared as follows on the day of transfection: siTOP1-1604 was diluted with an appropriate amount of Opti-MEM culture medium; Lipofectamine™ RNAiMAX (Lipo) transfection reagent was diluted with another appropriate amount of Opti-MEM culture medium and left to stand at room temperature for 5 min; and the two dilutions were uniformly mixed, left to stand at room temperature for 5 min, and then added to 24-well cell culture plates at 50 μL/well. PC-3 human prostate cancer cells were added to one of the 24-well cell culture plates at 4×10⁵ cells/well, the culture medium being 450 μL of F12K containing 10% FBS; HCT116 human colon cancer cells were added to the other of the 24-well cell culture plates at 3.5×10⁵ cells/well, the culture medium being 450 μL of IMDM containing 10% FBS. The cells were uniformly mixed with the siRNA. The final transfection volume was 500 μL, and the working concentrations of the siRNA were 10, 1.67, 0.27, 0.046, 0.0077, 0.0013, and 0.00021 nM. The control groups were Blank (Opti-MEM at the equal volume), Lipofectamine™ RNAiMAX (Lipo) transfection reagent, as well as 10 nM of siNC.

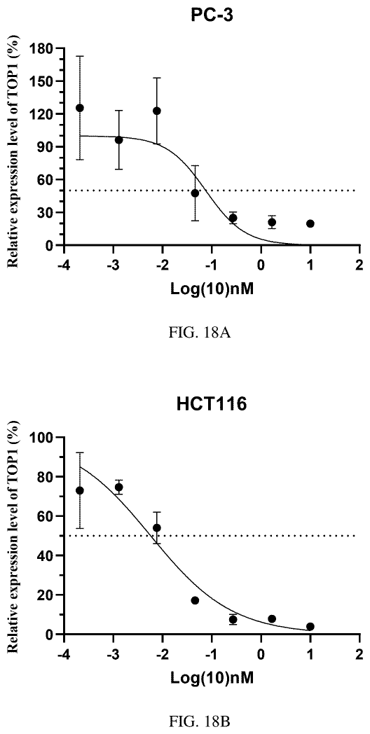

After 24 h of transfection, the cells were collected. The total cellular RNA was extracted according to the method in Example 2, and the level of TOP1 mRNA was detected. The results demonstrate that the relative expression levels of TOP1 in the two tumor cells were dose-dependent on siTOP1-1604 concentrations (Table 6). Nonlinear fitting was performed on the qPCR results using Graphpad software and IC50 of siTOP1 was calculated. The results demonstrate that siTOP1-1604 had an IC50 value of 0.075 nM in human prostate cancer cells and an IC50 value of 0.006 nM in human colon cancer cells relative to siNC (Table 7 and FIGS. 18A and 18B).

TABLE 6
Relative expression levels of TOP1 after the treatment
of different concentrations of siTOP1

| | | | PC-3 | | HCT116 | |
|---|---|---|---|---|---|---|
| | | | | Standard | | Standard |
| | | | Mean | deviation | Mean | deviation |
| Sample | | | value | (SD) | value | (SD) |
| Blank | | | 151.44 | 28.66 | 79.87 | 16.24 |
| Lipo | | | 127.51 | 45.26 | 74.98 | 6.63 |
| Lipo + siNC | 10 | nM | 100.00 | 4.90 | 100.00 | 7.45 |
| Lipo + | 10 | nM | 19.66 | 1.60 | 3.98 | 0.27 |
| siTOP1-1604 | 1.67 | nM | 21.05 | 5.85 | 7.91 | 0.89 |
| | 0.27 | nM | 24.94 | 5.51 | 7.57 | 2.61 |
| | 0.046 | nM | 47.64 | 25.21 | 17.23 | 1.67 |
| | 0.0077 | nM | 122.71 | 30.22 | 54.01 | 8.01 |
| | 0.0013 | nM | 96.21 | 26.87 | 74.72 | 3.56 |
| | 0.00021 | nM | 125.48 | 47.19 | 73.06 | 19.23 |

TABLE 7
IC50 (24 h) of siTOP1-1604 in PC-3 and HCT116

| | PC-3 | HCT116 |
|---|---|---|
| siTOP1-1604 | 0.075 nM | 0.006 nM |

### Example 5. Preparation of LNP-siTOP1

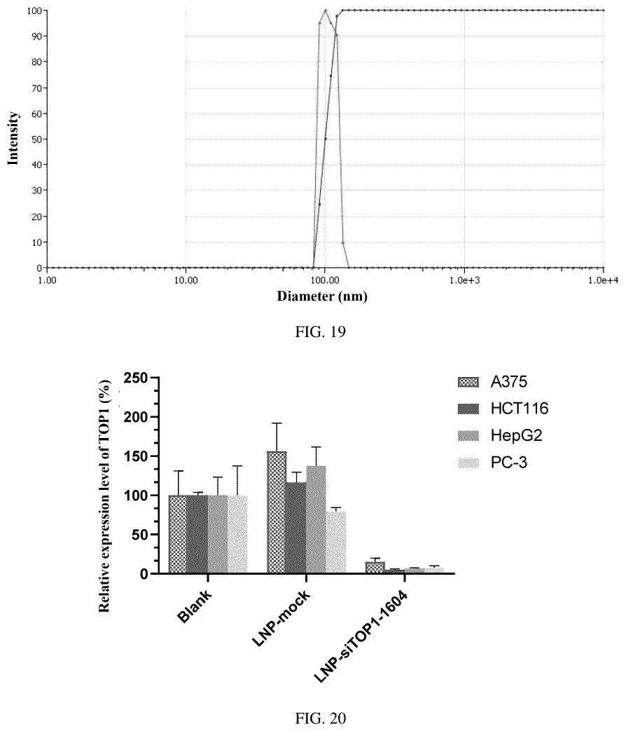

1) Preparation of stock solution for each component:
71.02 mg of SM-102 (Jenkem) was weighed and dissolved in 10 mL of anhydrous EtOH to give an SM-102 stock solution;
249.5 mg of M-DMG-2000 (Jenkem) was weighed and dissolved in 10 mL of anhydrous EtOH to give a PEG stock solution;
79.02 mg of DSPC (Sigma) was weighed and dissolved in 10 mL of anhydrous EtOH to give a DSPC stock solution;
38.66 mg of cholesterol (Sigma) was weighed and dissolved in 10 mL of anhydrous EtOH to give a cholesterol stock solution.
2) 2160 μL of the SM-102 stock solution, 64.8 μL of the PEG stock solution, 432 μL of the DSPC stock solution, and 1664 μL of the cholesterol stock solution were uniformly mixed to give an LNP/EtOH solution; 100 nmol siTOP1 (the siTOP1 screened from Example 1) was added to 6.5 mL of 50 mM citrate buffer (pH=4.0) to give an siTOP1/buffer solution.
3) Microfluidic preparation: The LNP/EtOH solution was used in channel 1, with a solution volume of 2 mL and a flow rate of 5 mL/min; the siTOP1/buffer solution was used in channel 2, with a solution volume of 6 mL and a flow rate of 15 mL/min; 4 mL of the microfluidic preparation solution was taken and dialyzed with PBS (pH=7.4) for 16 h to give LNP-siTOP1, resulting in a final siTOP1 concentration of 0.1 mg/mL. The effective particle size was detected using a NanoBrook 90Plus PALS particle size analyzer. Exemplarily, LNP-siTOP1-1604 had an effective particle size of 102.42 nm and a polydispersity coefficient of 0.064 (FIG. 19).

### Example 6: Detection of Inhibition of Gene Expression by LNP-siTOP1 in In Vitro Cell Experiment

On the day before transfection, HCT116 human colon cancer cells, HepG2 human liver cancer cells, and PC-3 human prostate cancer cells were seeded into 24-well plates at 1.25×10⁵ to 1.5×10⁵ cells/well, the culture media being 500 μL of IMDM, MEM, and F12K containing 10% FBS and 1% Penicillin-Streptomycin, respectively; A375 human melanoma cells were seeded into a 24-well plate at 6×10⁴ to 8×10⁴ cells/well, the culture medium being 500 μL of DMEM containing 10% FBS and 1% Penicillin-Streptomycin. After plating, the cell culture plates were placed in an incubator with 5% CO₂ at 37° C. for 24 h to allow the cells to adhere to the wall. Three replicate wells were made for each group. On the day of transfection, the culture media were each changed to 450 μL of culture medium without antibiotics (containing 10% FBS). Subsequently, 50 μL of LNP-siTOP1-1604 diluted with Opti-MEM was added to obtain a final transfection volume of 500 μL, and the working concentration of the siRNA was 50 nM. The control groups were Blank (Opti-MEM) and LNP-mock (diluted with Opti-MEM) without an siRNA. After transfection, the cells were cultured for another 48 h and collected. The total cellular RNA was extracted according to the method in Example 2, and the level of TOP1 mRNA was detected. The results demonstrate that LNP-siTOP1-1604 prepared in Example 5 was capable of inhibiting the expression levels of TOP1 mRNA in various tumor cells in the in vitro experiment. Specifically, after 48 h of transfection, TOP1 reduced by 84.7% in A375, 95.0% in HCT116, 93.2% in HepG2, and 92.4% in PC-3, compared to Blank (Table 8 and FIG. 20).

TABLE 8
Detection of the inhibitory effect of LNP-siTOP1-1604 on the gene in tumor cells

| | Blank | | LNP-mock | | LNP-siTOP1-1604 | |
|---|---|---|---|---|---|---|
| | Mean | Standard | Mean | Standard | Mean | Standard |
| Cell | value | deviation (SD) | value | deviation (SD) | value | deviation (SD) |
| A375 | 100.00 | 30.90 | 156.09 | 35.98 | 15.31 | 4.57 |
| HCT116 | 100.00 | 3.87 | 116.26 | 13.25 | 5.01 | 1.16 |
| HepG2 | 100.00 | 23.25 | 137.61 | 24.04 | 6.81 | 0.82 |
| PC-3 | 100.00 | 37.63 | 79.44 | 4.79 | 7.58 | 2.56 |

### Example 7: Inhibitory Effect of LNP-siTOP1 on Human Prostate Cancer Cell Proliferation

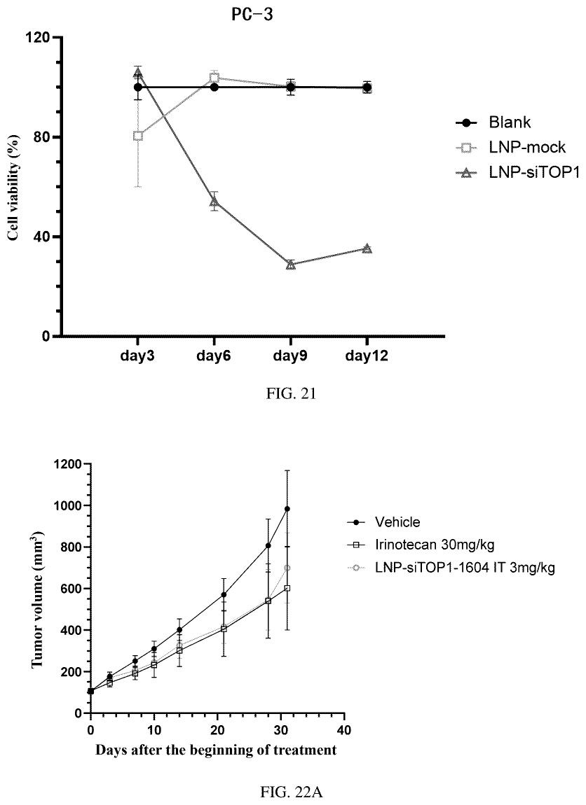

On the day before transfection, PC-3 human prostate cancer cells were seeded into a 96-well cell culture plate at 1×10⁴ cells/well, the culture medium being F12K containing 10% FBS and 1% Penicillin-Streptomycin, and after plating, the cell culture plate was placed in an incubator with 5% CO₂ at 37° C. for 24 h to allow the cells to adhere to the wall. 4 replicate wells were made for each group in the 96-well plate. On the day of transfection, the culture medium was changed to an F12K culture medium without antibiotics (containing 10% FBS). LNP-siTOP1-1604 diluted with Opti-MEM culture medium was added, and the working concentration of the siRNA was 200 nM. The control groups were Blank (Opti-MEM at the equal volume) and LNP-mock (diluted with Opti-MEM) without an siRNA. The cell culture plate was placed in the incubator with 5% CO₂ at 37° C., and the cells continued to be cultured. The cell viability was detected every 72 h according to the method in Example 3. The results demonstrate that LNP-siTOP1-1604 was capable of inhibiting PC-3 proliferation, and the lowest viable cell count was found in the LNP-siTOP1-1604 group on day 9 after transfection, which was 71.2% lower than that in the Blank group (Table 9 and FIG. 21).

TABLE 9
Inhibitory effect of a single transfection of
LNP-siTOP1-1604 on PC-3 cell proliferation

| | Blank | | LNP-mock | | LNP-siTOP1-1604 | |
|---|---|---|---|---|---|---|
| | Mean | Standard | Mean | Standard | Mean | Standard |
| | value | deviation (SD) | value | deviation (SD) | value | deviation (SD) |
| day 3 | 100.00 | 4.98 | 80.52 | 20.48 | 105.97 | 2.59 |
| day 6 | 100.00 | 0.47 | 103.76 | 2.97 | 54.20 | 3.86 |
| day 9 | 100.00 | 3.18 | 100.32 | 1.97 | 28.83 | 1.84 |
| day 12 | 100.00 | 2.36 | 99.47 | 0.88 | 35.29 | 0.60 |

### Example 8: Pharmacodynamic Study of LNP-siTOP1 in Prostate Cancer Mouse Model

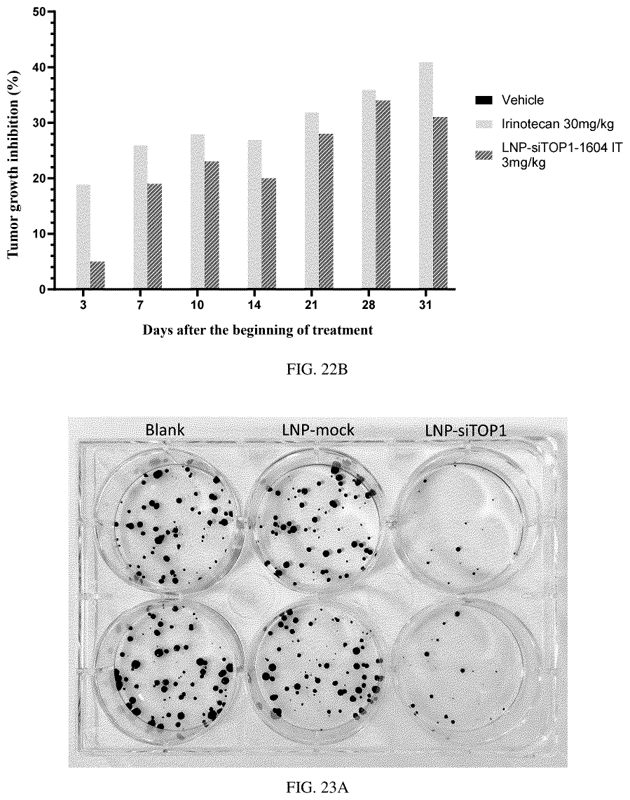

In the in vivo pharmacodynamic assay, male NCG mice aged 6-8 weeks were used as the study subjects; PC-3 human prostate cancer cells in the logarithmic growth phase were inoculated subcutaneously to the right flank of the test animals; and the mice were grouped with 6 mice per group when the tumors grew to an average volume of 100-150 mm³. The day of grouping was recorded as DO. The experimental groups, the administration doses, and the administration frequency were as follows: (1) vehicle group (Vehicle), (2) irinotecan group (Irinotecan), which were administered once on each of D0 and D14 at 30 mg/kg, and (3) LNP-siTOP1-1604 group, which was intratumorally injected once on each of D0, D15, and D23 at 3 mg/kg. The tumor volume and the mouse body weight were measured and recorded periodically. Animal test results demonstrate that LNP-siTOP1-1604 had a certain inhibitory effect on tumor growth compared to the vehicle control, and the tumor growth inhibition (TGI) on D28 reached 28 (FIGS. 22A and 22B).

### Example 9: Toxicities of LNP-siTOP1 and Irinotecan on Human Colon Cancer Cells

On the day before transfection, HCT116 human colon cancer cells were seeded into a 96-well cell culture plate at 5000 cells/well, the culture medium being IMDM containing 10% FBS and 1% Penicillin-Streptomycin, and after plating, the cell culture plate was placed in an incubator with 5% CO₂ at 37° C. for 24 h to allow the cells to adhere to the wall. 3 replicate wells were made for each group. On the day of transfection, the culture medium was changed to an IMDM culture medium without antibiotics (containing 10% FBS). LNP-siTOP1-1604 diluted with Opti-MEM culture medium was added, and the working concentrations of the siRNA were 500, 200, 100, 50, 20, and 5 nM. The control groups were Blank (Opti-MEM at the equal volume) and the positive control drug, irinotecan, at concentrations of 500, 200, 100, 50, 20, 5, and 1 μM. The cell culture plate was placed in the incubator with 5% CO₂ at 37° C., and the cells were cultured for another 72 h. The cell viability was detected according to the method in Example 3 (Table 10). Fitting was performed using Graphpad software. The results demonstrate that IC50 of LNP-siTOP1-1604 was 25.90 nM, significantly lower than IC50 of irinotecan (Table 11).

TABLE 10
Inhibitory Effects of different concentrations of
LNP-siTOP1 and irinotecan on HCT116 proliferation

Cell viability (%)

| | | Standard |
|---|---|---|
| | Mean | deviation |
| Sample | value | (SD) |

| Blank | | | | 100.00 | 1.55 |
|---|---|---|---|---|---|
| | LNP-siTOP1-1604 | 500 | nM | 24.70 | 0.31 |
| | | 200 | nM | 30.01 | 0.57 |
| | | 100 | nM | 35.30 | 1.02 |
| | | 50 | nM | 37.81 | 0.42 |
| | | 20 | nM | 47.96 | 0.80 |
| | | 5 | nM | 74.13 | 5.32 |
| | Irinotecan | 500 | μM | 11.87 | 0.36 |
| | | 200 | μM | 8.18 | 0.01 |
| | | 100 | μM | 9.69 | 0.14 |
| | | 50 | μM | 16.94 | 0.67 |
| | | 20 | μM | 31.84 | 3.18 |
| | | 5 | μM | 55.44 | 4.03 |
| | | 1 | μM | 108.71 | 4.69 |

TABLE 11
IC50 of LNP-siTOP1-1604 and irinotecan in HCT116 (72 h)

| | LNP-siTOP1-1604 | Irinotecan |
|---|---|---|
| HCT116 | 25.90 nM | 9.328 μM |

### Example 10: Inhibition of Colony Formation of HCT116 Human Colon Cancer Cells by LNP-siTOP1

On the day before transfection, HCT116 human colon cancer cells were seeded into a 6-well cell culture plate at 5×10⁵ cells/well, the culture medium being 2 mL of IMDM containing 10% FBS and 1% Penicillin-Streptomycin, and after plating, the cell culture plate was placed in an incubator with 5% CO₂ at 37° C. for 24 h to allow the cells to adhere to the wall. On the day of transfection, the culture medium was changed to 1.8 mL of culture medium without antibiotics (containing 10% FBS). Subsequently, 200 μL of LNP-siTOP1-1604 diluted with Opti-MEM was added to obtain a final transfection volume of 2 mL, and the working concentration of the siRNA was 50 nM. The control groups were Opti-MEM and LNP-mock (diluted with Opti-MEM) without an siRNA. The cells were cultured for another 24 h, then the cells were digested with 0.25% trypsin-EDTA and seeded again into a 6-well cell culture plate at 1000 cells/well with two replicate wells made for each group. The cell culture plate was placed in the incubator with 5% CO₂ at 37° C., and the cells were cultured for another 2 weeks, with periodic culture medium replacement.

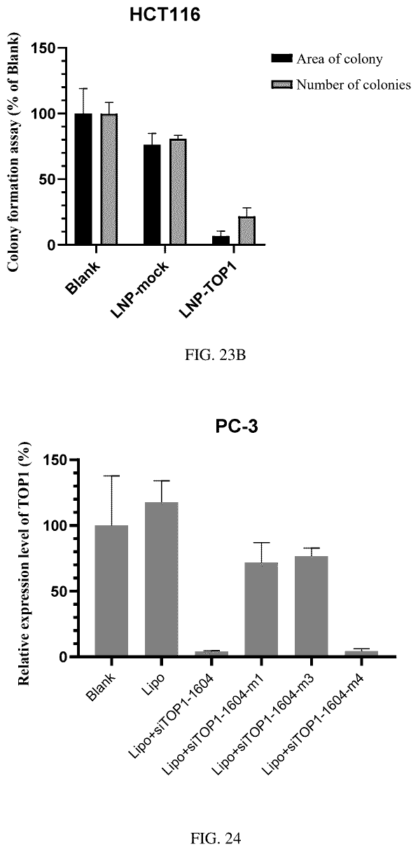

After the experiment was completed, the culture medium was removed and the plate was washed with PBS. 1 mL of 0.1% crystal violet staining solution was added, and the cells were stained at room temperature for 10 min. After the unbound stains were washed off with PBS, photographing was performed using a camera and an imager, and the numbers as well as the areas of colonies in each group were counted using ImageJ software. The results demonstrate that the numbers of colonies and the areas of colonies in the LNP-siTOP1-1604 group were significantly less than those in the control groups (Table 12 and FIGS. 23A and 23B), indicating that LNP-siTOP1-1604 is capable of inhibiting the growth of HCT116 human colon cancer cells.

TABLE 12
Inhibitory effect of LNP-siTOP1-1604 on HCT116

| | | | |
|---|---|---|---|
| 1 | 2 | Mean value | % of Blank |

Area of colony

| | | | | |
|---|---|---|---|---|
| Blank | 0.064 | 0.084 | 0.074 | 100.00 |
| LNP-mock | 0.052 | 0.061 | 0.0565 | 76.35 |
| LNP-siTOP1-1604 | 0.003 | 0.007 | 0.005 | 6.76 |

Number of colonies

| | | | | |
|---|---|---|---|---|
| Blank | 141 | 125 | 133 | 100.00 |
| LNP-mock | 105 | 110 | 107.5 | 80.83 |
| LNP-siTOP1-1604 | 35 | 23 | 29 | 21.80 |

### Example 11: Inhibitory Effects of Modified Sequences of siTOP1-1604 on Gene

The sequence of siTOP1-1604 was chemically modified, and the modified sequences are shown in Table 13 below.

TABLE 13
Modified sequences of siTOP1-1604 Modification

| Name | Sense strand | Antisense strand | method |
|---|---|---|---|
| siTOP1- | 5′- | 5′- | Methylation |
| 1604-m1 | G(mU)G(mU)GGA(mC)AAGA(mU) | (mU)(mU)(mC)(mC)GGA(mU) | |
| | (mC)(mC)GGAAdTdT-3′ (SEQ ID | (mC)(mU)(mU)G(mU)(mC)(mC) | |
| | NO: 51) | A(mC)A(mC)dTdT-3′ (SEQ ID | |
| | | NO: 52) | |
| siTOP1- | 5′-(mG)-(mU)- | 5′-(mU)-(mU)- | Methylation, |
| 1604-m3 | (mG)(mU)(mG)(mG)(mA)(mC)(mA) | (mC)(mC)(mG)(mG)(mA)(mU) | fluorination, and |
| | (fA)(fG)(fA)(mU)(mC)(mC)(mG) | m(C)(mU)(mU)(mG)(mU)(mC) | phosphorothioate |
| | (mG)(mA)(mA)dTdT-3′ (SEQ ID | (mC)(mA)(mC)-(mA)-(mC)dTdT- | |
| | NO: 53) | 3′ (SEQ ID NO: 54) | |
| siTOP1- | 5′- | 5′- | Pseudouridine |
| 1604-m4 | G(ΨU)G(ΨU)GGACAAGA(ΨU)CC | (ΨU)(ΨU)CCGGA(ΨU)C(ΨU) | |
| | GGAAdTdT-3′ (SEQ ID NO: 55) | (ΨU)G(ΨU)CCACACdTdT- | |
| | | 3′ (SEQ ID NO: 56) | |

m represents a methylation modification (2′-O-methyl); f represents a fluorination modification (2′-deoxy-2′-fluoro); - represents a phosphorothioate modification (p); Ψ represents a pseudouridine modification.

PC-3 human prostate cancer cells were transfected with the modified siTOP1 according to the method in Example 2 at a siRNA working concentration of 50 nM. TOP1 mRNA levels were detected 48 h after the transfection. The control groups were Blank (Opti-MEM at the equal volume), Lipofectamine™ RNAiMAX (Lipo) transfection reagent, and the positive control unmodified siTOP-1604. The experimental results demonstrate that pseudouridine-modified siTOP1 inhibited the expression of TOP1 mRNA to 4.3% compared to the control groups (Table 14 and FIG. 24).

TABLE 14
Inhibitory effects of modified sequences
of siTOP1-1604 on the gene

Relative expression level

| | | Standard |
|---|---|---|
| | Mean | deviation |
| PC-3 | value | (SD) |
| Blank | 100.00 | 37.63 |
| Lipo | 117.67 | 16.36 |
| Lipo + siTOP1-1604 | 4.03 | 0.76 |
| Lipo + siTOP1-1604-m1 | 71.77 | 15.22 |
| Lipo + siTOP1-1604-m3 | 76.66 | 6.16 |
| Lipo + siTOP1-1604-m4 | 4.30 | 1.86 |

The preferred embodiments of the present invention are described in detail above, which, however, are not intended to limit the present invention. Within the scope of the technical concept of the present invention, various simple modifications can be made to the technical solution of the present invention, all of which will fall within the protection scope of the present invention. In addition, it should be noted that the various specific technical features described in the above specific embodiments can be combined in any suitable manner in which the features do not contradict each other. In order to avoid unnecessary repetition, such combinations will not be illustrated separately in the present invention.

1. An interfering RNA targeting TOP1 gene, wherein a target site sequence of the interfering RNA comprises a nucleotide sequence set forth in any one or two or more of SEQ ID NOs: 1-15.

2. The interfering RNA according to claim 1, comprising one or a combination of two or more of an siRNA, a dsRNA, an shRNA, an aiRNA, or an miRNA.

3. The interfering RNA according to claim 1, further comprising an over-hang.

4. The interfering RNA according to claim 3, wherein the over-hang is located at the 3′ terminus of a sense strand and/or an antisense strand of the interfering RNA.

5. The interfering RNA according to claim 3, wherein the over-hang is a deoxynucleoside.

6. The interfering RNA according to claim 1, comprising a sense strand and/or an antisense strand,
the sense strand comprises a nucleotide sequence set forth in any one or two or more of SEQ ID NOs: 16-30;
the antisense strand comprises a nucleotide sequence set forth in any one or two or more of SEQ ID NOs: 31-45.

7. The interfering RNA according to claim 1, further comprising at least one modification, wherein the modification comprises modifications on chemical structures of a base, a sugar ring, and/or a phosphate,
the modification on a base comprises, but is not limited to, a pyrimidine modification at position 5, a purine modification at position 8, and/or a 5-bromouracil substitution;
the modification on a sugar ring comprises, but is not limited to, a substitution of 2′-OH with H, OZ, Z, halo, SH, SZ, NH₂, NHZ, NZ₂, or CN group, wherein Z is an alkyl group;
the modification on a phosphate backbone comprises, but is not limited to, a phosphorothioate modification.

8. The interfering RNA according to claim 7, wherein the modification further comprises nucleotides having inosine, queuosine, xanthine, 2′-methylribose, a non-natural phosphodiester bond (e.g., methylphosphonate and phosphorothioate), and/or a peptide.

9. A delivery system for the interfering RNA according to claim 1, comprising the interfering RNA according to claim 1 and a vector.

10. The delivery system according to claim 9, wherein the vector is a viral vector or a non-viral vector;
the viral vector comprises one or a combination of two or more of a lentivirus vector, a retrovirus vector, an adenovirus vector, an adeno-associated virus vector, a poxvirus vector, or a herpesvirus vector;
the non-viral vector comprises any one or a combination of two or more of a liposome, a lipid nanoparticle, a polymer, a polypeptide, an antibody, an aptamer, or N-acetylgalactosamine (GalNAc).

11. The delivery system according to claim 10, wherein the lipid nanoparticle/liposome comprises one or a combination of two or more of a cationic lipid, a neutral lipid, a polyethylene glycol lipid, a steroidal lipid, or an anionic lipid.

12. The delivery system according to claim 11, wherein the cationic lipid comprises one or a combination of two or more of stearamide (SA), lauryltrimethylammonium bromide, hexadecyltrimethylammonium bromide, myristyltrimethylammonium bromide, dimethyldioctadecylammonium bromide (DDAB), [(4-hydroxybutyl)azanediyl]di(hexane-6,1-diyl) bis(2-hexyldecanoate) (ALC-0315), 1,2-dioleoyloxy-3-(trimethylammonium)propane (DOTAP), 1,2-di-(9Z-octadecenoyl)-3-trimethylammonium-propane and 1,2-dihexadecanoyl-3-trimethylammonium-propane, 30-[N—(N′,N′-dimethylaminoethane)-carbamoyl]cholesterol (DC-cholesterol), dimethyldioctadecylammonium (DDA), 1,2-dimyristoyl-3-trimethylammonium propane (DMTAP), dipalmitoyl(C16:0)trimethylammonium propane (DPTAP), distearoyltrimethylammonium propane (DSTAP), N-[1-(2,3-diallyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium propane (DODAP), 1,2-dilinoleyloxy-3-dimethylaminopropane (DLinDMA), 1,2-ditetradecanoyl-3-dimethylammonium-propane, 1,2-dihexadecanoyl-3-dimethylammonium-propane and 1,2-dioctadecanoyl-3-dimethylammonium-propane, 1,2-dioleoyl-c-(4′-trimethylammonium)-butanoyl-sn-glycerol (DOTB), dioctadecylamidoalanyl spermine, SAINT-2, polycationic lipid 2,3-dioleoyloxy-N-[2(spermine-carboxamido)ethyl]-N,N-dimethyl-1-propanaminium trifluoroacetate (DOSPA),

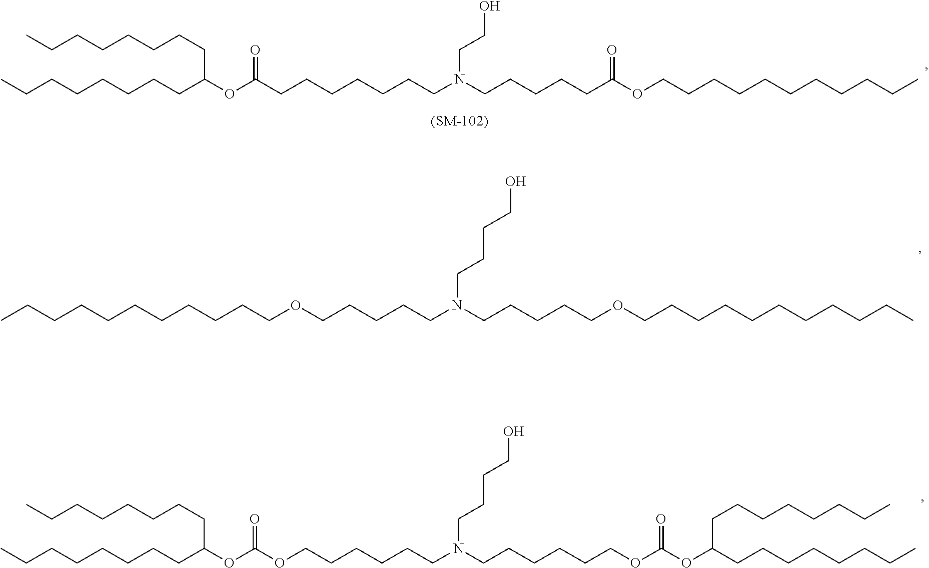

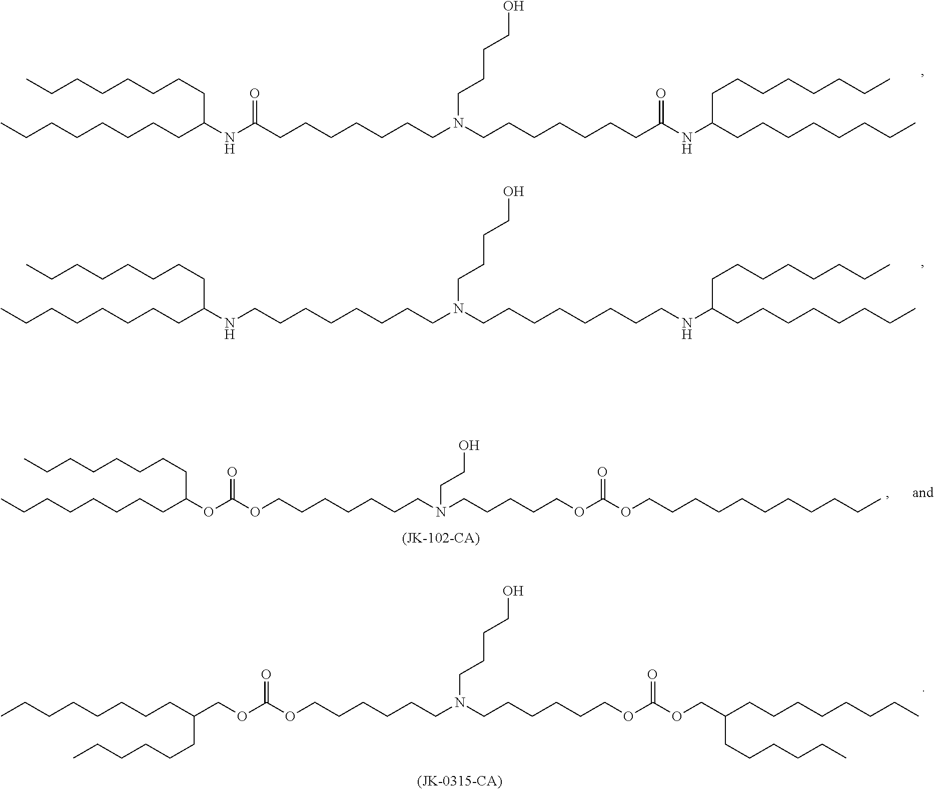

13. The delivery system according to claim 11, wherein the neutral lipid comprises one or a combination of two or more of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), or distearoylphosphatidylethanolamine (DSPE).

14. The delivery system according to claim 11, wherein the polyethylene glycol lipid comprises one or a combination of two or more of 2-[(polyethylene glycol)-2000]-N,N-ditetradecylacetamide (ALC-0159), 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disterol glycerol (PEG-DSG), PEG-dipalmitoyl, PEG-dioleyl, PEG-distearyl, PEG-diacylglycerol amide (PEG-DAG), PEG-dipalmitoyl phosphatidylethanolamine (PEG-DPPE) or PEG-1,2-dimyristoyloxypropyl-3-amine (PEG-c-DMA), 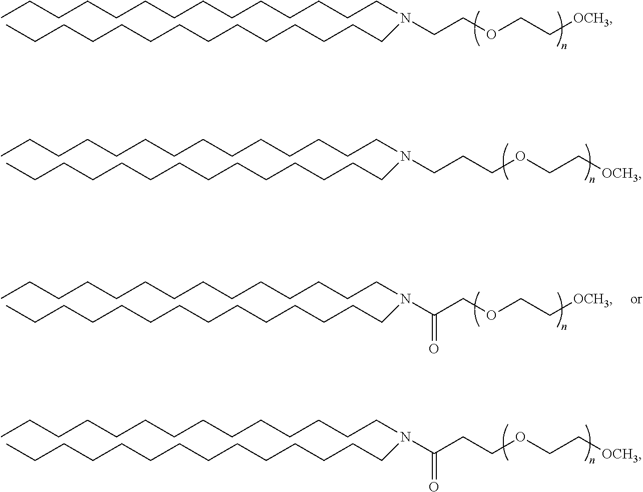
wherein n is selected from integers of 20-300.

15. The delivery system according to claim 11, wherein the polyethylene glycol lipid is a polyethylene glycol lipid with a single molecular weight,
the polyethylene glycol lipid comprises:

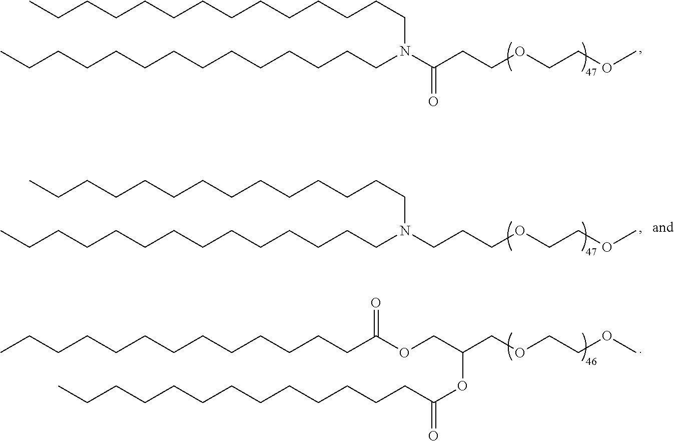

16. The delivery system according to claim 11, wherein the cationic lipid is a steroid-cationic lipid compound
having a structure of one or two or more of the following:

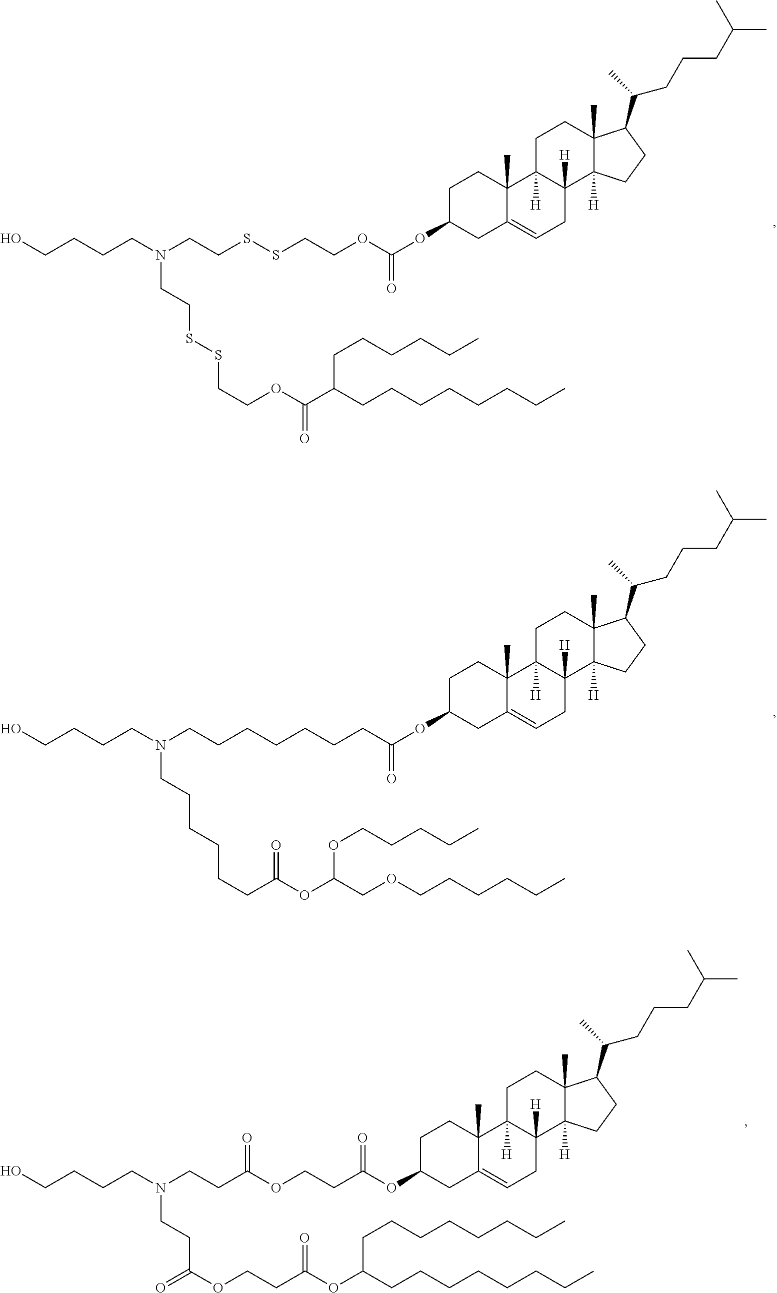

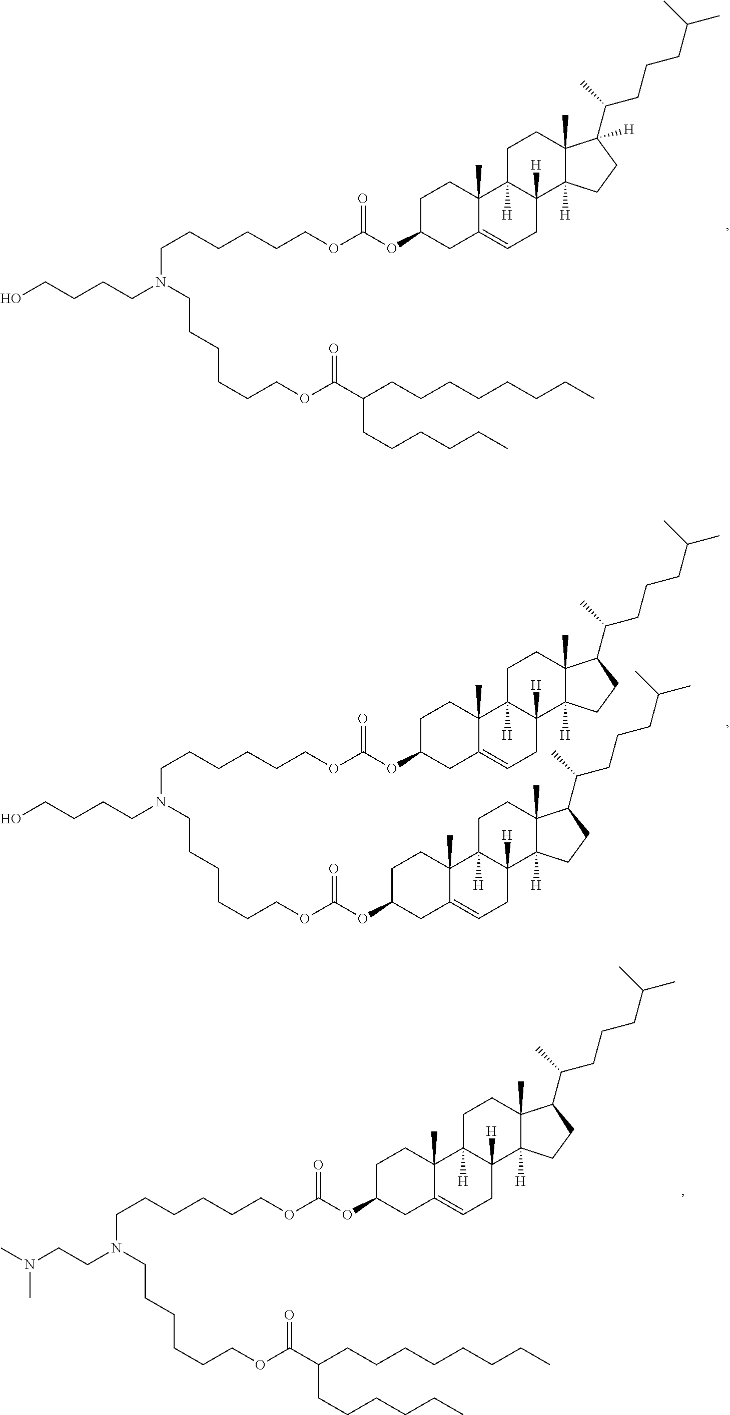

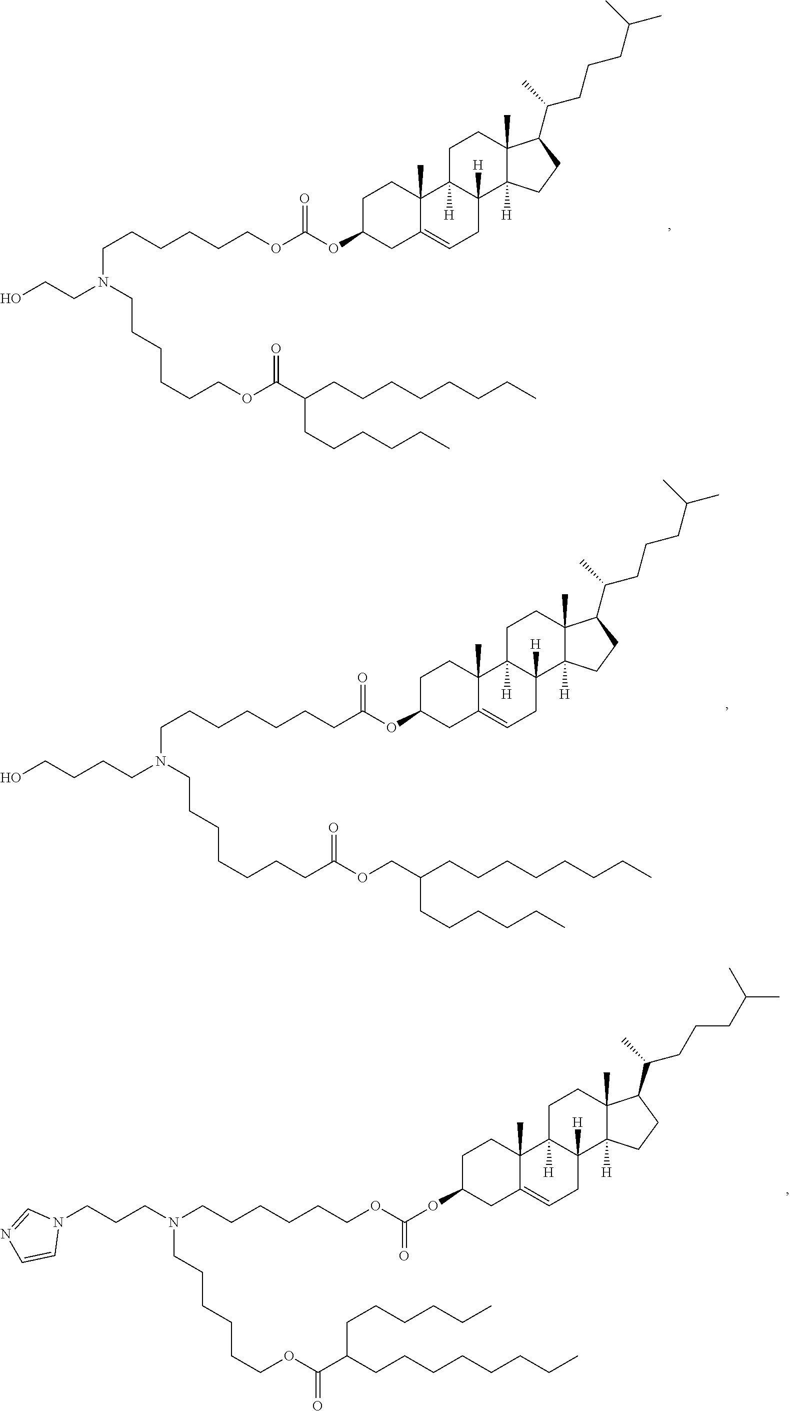

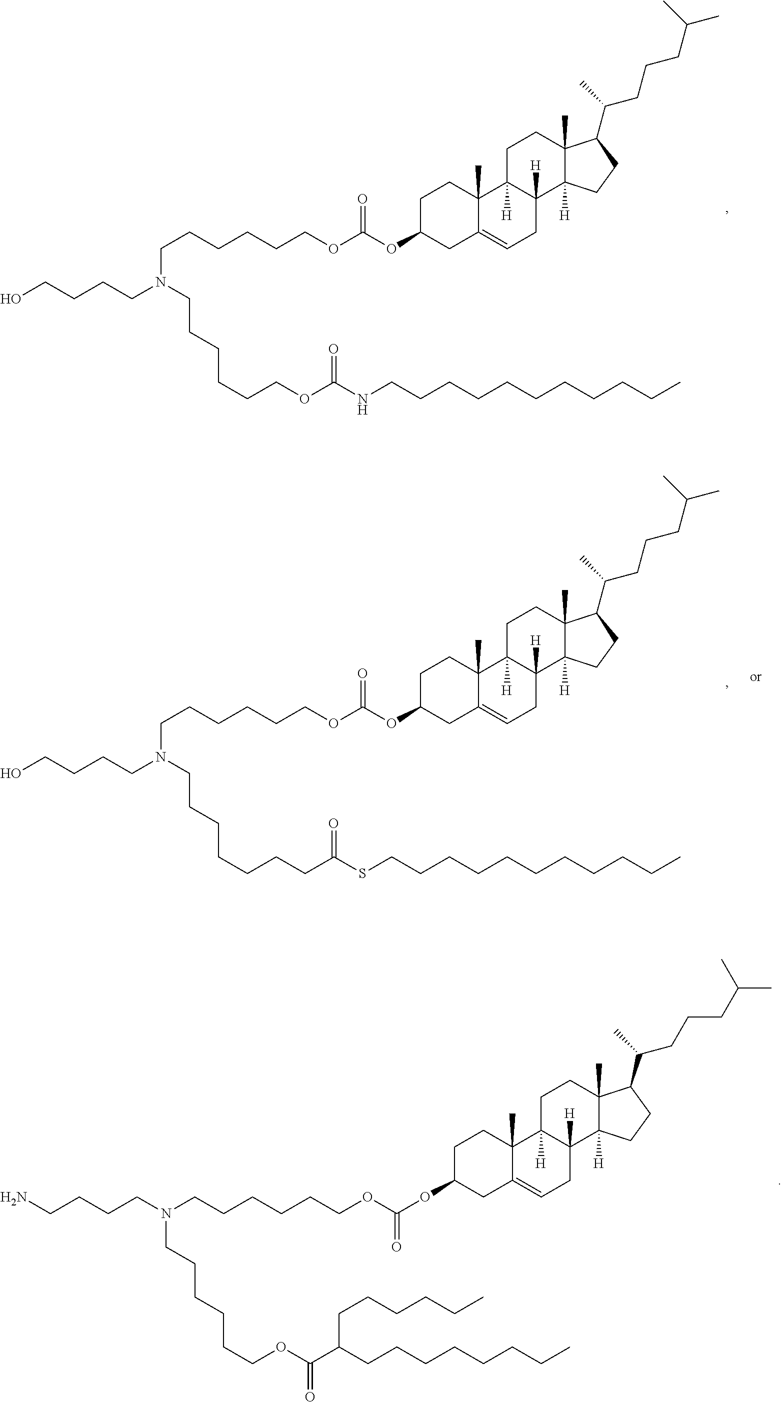

17. The delivery system according to claim 11, wherein the anionic liposome comprises one or a combination of two or more of dioleoyl phosphatidylglycerol or dioleoyl phosphatidylethanolamine.

18. The delivery system according to claim 11, wherein the steroidal lipid comprises one or a combination of two or more of avenasterol, β-sitosterol, brassicasterol, ergocalciferol, campesterol, cholestanol, cholesterol, coprostanol, dehydrocholesterol, desmosterol, dihydroergocalciferol, dihydrocholesterol, dihydroergosterol, dinosterol, epicholesterol, ergosterol, fucosterol, hexahydrolumisterol, hydroxycholesterol, lanosterol, lumisterol, saringosterol, sitostanol, sitosterol, stigmastanol, stigmasterol, cholic acid, glycocholic acid, taurocholic acid, deoxycholic acid, or lithocholic acid.

19.-32. (canceled)

33. A method for inhibiting TOP1 expression, comprising adding the interfering RNA according to claim 1, or the delivery system comprising the interfering RNA thereof.

34. A method for treating a TOP1 expression-associated disease, comprising administering to a subject a therapeutically effective amount of the interfering RNA according to claim 1, or the delivery system comprising the interfering RNA thereof.

35.-37. (canceled)
